(19) United States
(12) Patent Application Publication
(10) Pub. No.: US 2025/0261843 A1
(43) Pub. Date: Aug. 21, 2025

(54) LOWER GENITAL TRACT ACCESS DEVICES, METHODS AND KITS

(71) Applicant: MEDICINES360, San Francisco, CA (US)

(72) Inventors: Rob DECKMAN, San Bruno, CA (US); Mitchell David CREININ, Davis, CA (US); Dominic PERALTA, San Mateo, CA (US); Chinmay DEODHAR, Hannover (DE); Tonatiuh ESQUIVEL, Orland, CA (US); Julie GROH, Petaluma, CA (US); Adeline MASBRATA, Fremont, CA (US); Andrea Ildiko OLARIU, Fremont, CA (US); Andrew Ivan POUTIATINE, Mill Valley, CA (US); Nicanor Asinas DOMINGO, Milpitas, CA (US)

(73) Assignee: MEDICINES360, San Francisco, CA (US)

(21) Appl. No.: 19/052,540

(22) Filed: Feb. 13, 2025

Related U.S. Application Data
(60) Provisional application No. 63/632,025, filed on Apr. 10, 2024.
(60) Provisional application No. 63/554,377, filed on Feb. 16, 2024.

(51) Int. Cl. A61B 1/303

(52) U.S. Cl. CPC A61B 1/303

(57) ABSTRACT

Lower genital tract access devices, methods, systems and kits Disclosed are devices, methods, systems and kits for obtaining biological tissue samples. Also disclosed are hysteroscopy or colposcopy devices that eliminate the need for a separate speculum and that improves patient comfort.

This application claims the benefit of U.S. Provisional Patent Application Nos. 63/632,025, filed Apr. 10, 2024, and 63/554,377, filed Feb. 16, 2024, which applications are incorporated herein in their entirety by reference.

### BACKGROUND

Field of Invention: Devices for lower genital tract access. Devices for use in colposcopy procedures, hysteroscopy procedures and for pelvic examinations.

Background: For context, the female reproductive anatomy 50 is illustrated in FIG. 1A. The opening of the female reproductive anatomy 50 includes the vulva 52. The vulva describes the structures comprising the external genitalia. The vagina 54 is an elastic, muscular part of the female genital tract. In humans the vagina extends from the vestibule to the female cervix 56. The female cervix 56 is the lower part of the uterus 58. Fallopian tubes 57 and ovaries 59 also form part of the female reproductive anatomy.

Colposcopy is a medical procedure performed to provide a close examination of a female cervix 56, vagina 54 and vulva 52. The colposcopy procedure, illustrated in FIG. 1B, is typically performed to detect signs of disease. A colposcopy procedure uses a colposcope. The colposcope devices 100 allows examination of the female cervix 56 from a location external to the patient under illumination of various low-power magnifications using a light beam 102. Colposcope devices 100 include a binocular microscope and light source. The light source can incorporate a beam splitter to allow attachment of a still or video camera.

Hysteroscopy is a medical procedure performed to examine the inside of the female cervix 56 and uterus 58 using a thin, lighted, flexible tube called a hysteroscope 150 as shown in FIG. 1C. Unlike the colposcope, the hysteroscope is used to access the anatomy inside the genitalia.

A variety of tools can also be used with the colposcope or hysteroscope, including, for example, an endocervical curette, cervical biopsy pump and cervical swabs, and speculum 110. The speculum 110 is used to provide access and/or visual access to the cervix. The speculum is a metal or plastic instrument and is separate from the colposcope or hysteroscope. The speculum is used to dilate an orifice or canal in a body to allow inspection. Speculums often cause the patient pain during use. The vaginal speculum is used during pelvic examinations and can be used to allow a healthcare provider to insert, for example, a small brush, swab or spatula to gently remove cells from the cervix and the back of the vagina.

What is needed are devices, systems and methods for lower genital tract access capable of performing obtaining biological material samples. Also what is needed are devices, systems and methods for hysteroscopy or colposcopy procedures that eliminate the need for a speculum, improves patient comfort and allows the procedure to be performed quickly and remotely.

### SUMMARY

Disclosed are devices, systems and methods for obtaining biological tissue samples. Also disclosed are hysteroscopy or colposcopy devices that eliminate the need for a speculum and that improves patient comfort. Additionally the devices, systems and methods allows the hysteroscopy or colposcopy procedure to be performed more quickly and remotely. The devices, systems and methods are further optimizable for use in a remote or mobile setting, including use in a home environment. Additionally, the devices, systems and methods can be used by an individual on herself or by an untrained individual to provide broader public access to healthcare. Moreover the devices, systems and methods can be used for delivery of therapeutic applications including but not limited to drug delivery, laser delivery, heat or cold delivery, etc.

The disclosed devices can comprise: a housing having a proximal housing end and a distal housing end, wherein a first portion of the housing extending from the proximal housing end defines a first elongated member and a second portion of the housing extending from the first elongated member to the distal housing end fines a second elongated member; one or more channels defined within an interior of the housing; an expandable distal housing end; a distal end diameter controller wherein the distal end diameter controller is operable to control a diameter of a distal end of the housing; and a conical elongated housing aperture with a first conical elongated housing aperture diameter and a second conical elongated housing aperture diameter, larger than the first conical elongated housing aperture diameter.

Another configuration of the disclosed devices comprises: a housing having a proximal housing end and a distal housing end, wherein a first portion of the housing extending from the proximal housing end defines a first elongated member and a second portion of the housing extending from the first elongated member to the distal housing end fines a second elongated member wherein the first elongated member has an expandable distal housing portion having a first diameter in a closed configuration, a proximal housing portion having a second diameter and an intermediate housing portion positioned between the distal housing portion and the proximal housing portion having a third diameter less than the first diameter; one or more channels defined within an interior of the housing; a distal end diameter controller wherein the distal end diameter controller is operable to control a diameter of a distal end of the housing; and a housing aperture positioned operable to provide access to an interior of the housing.

Still another configuration of the disclosed devices comprises: a housing having a proximal housing end and a distal housing end, wherein a first portion of the housing extending from the proximal housing end defines a first elongated member and a second portion of the housing extending from the first elongated member to the distal housing end fines a second elongated member; one or more channels defined within an interior of the housing; a distal end diameter controller wherein the distal end diameter controller is operable to control a diameter of a distal end of the housing; an expandable distal housing end; and one or more electronic components operable to transmit information captured by the device to a secondary electronic device.

Yet another configuration of the disclosed devices comprises: a housing having a proximal housing end and a distal housing end, wherein a first portion of the housing extending from the proximal housing end defines a first elongated member and a second portion of the housing extending from the first elongated member to the distal housing end fines a second elongated member; one or more channels defined within an interior of the housing; a distal end diameter controller wherein the distal end diameter controller is operable to control a diameter of a distal end of the housing and further wherein the distal end diameter control is selected from a rotatable member, a slidable button, a pressable button, a retractable sheath trigger and a retractable s heath lever; and an expandable distal housing end.

Systems are also disclosed. Suitable systems comprise software operable to: communicate with the devices disclosed herein to control an operation of the device; capture one or more images of interest via an image capture device and store the one or more images of interest in a database; and control a zoom function of an optical lens.

Additionally, methods of using the disclosed devices are also disclosed. The methods comprise: providing an access device such as the disclosed devices; inserting the access device into a vagina; controlling an operation of the access device via a distal end diameter controller; and activating a tool.

Kits are also included. Suitable kits include an access device and one or more of a camera, an endocervical curette, a cervical biopsy pump, cervical swab, sample collection device, and vial.

Both the foregoing general description and the following detailed description are exemplary and explanatory only and are not restrictive of the disclosed embodiments, as claimed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.
U.S. Pat. No. 4,134,637 A issued Jan. 16, 1979 by Leisegang;
U.S. Pat. No. 4,232,933 A issued Nov. 11, 1980 by Nakahashi;
U.S. Pat. No. 4,491,131 A issued Jan. 1, 1985 by Vassiliadis;
U.S. Pat. No. 4,652,103 A issued Mar. 24, 1987 by Klabes;
U.S. Pat. No. 6,031,619 A issued Feb. 29, 2000 by Wilkens et al.;
U.S. Pat. No. 6,101,408 A issued Aug. 8, 2000 by Craine et al.;
U.S. Pat. No. 6,147,705 A issued Nov. 14, 2000 by Krauter et al.;
U.S. Pat. No. 6,277,067 B1 issued Aug. 21, 2001 by Blair;
U.S. Pat. No. 6,359,644 B1 issued Mar. 19, 2002 by Salvati;
U.S. Pat. No. 6,766,184 B2 issued Jul. 20, 2004 by Utzinger et al.;
U.S. Pat. No. 8,187,180 B2 issued May 29, 2012 by Pacey;
U.S. Pat. No. 9,687,197 B2 issued Jun. 27, 2017 by Yetik et al.;
U.S. Pat. No. 10,028,649 B2 issued Jul. 24, 2018 by Salvati et al.;
U.S. Pat. No. 10,127,665 B1 issued Nov. 13, 2018 by Ding et al.;
U.S. Pat. No. 10,162,935 B2 issued Dec. 25, 2018 by Pidathala et al.;
U.S. Pat. No. 10,857,151 B1 issued Dec. 8, 2020 by Miller;
U.S. Pat. No. 11,553,981 B2 issued Jan. 17, 2023 by Wood et al.;
U.S. Pat. No. 11,583,177 B2 issued Feb. 21, 2023 by Landesman et al.;
U.S. Pat. No. 11,619,548 B2 issued Apr. 4, 2023 by Balas et al.; and
U.S. Pat. No. 11,805,994 B2 issued Nov. 7, 2023 to Ramanujam et al.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates a portion of the female anatomy;

FIG. 1B illustrates a colposcope in use;

FIG. 1C illustrates a hysteroscope in use;

FIGS. 2A-P illustrate an embodiment of an access device for use in a colposcopy or hysteroscopy procedure;

FIGS. 3A-P illustrate another embodiment of an access device for use in a colposcopy or hysteroscopy procedure;

FIGS. 4A-F illustrate another embodiment of an access device for use in a colposcopy or hysteroscopy procedure;

FIGS. 5A-F illustrate another embodiment of an access device for use in a colposcopy or hysteroscopy procedure;

FIGS. 6A-F illustrate another embodiment of an access device for use in a colposcopy or hysteroscopy procedure;

FIGS. 7A-D illustrate distal end configurations of access devices for use in a colposcopy or hysteroscopy procedures;

FIGS. 8A-J illustrate additional distal end configurations of access devices for use in a colposcopy or hysteroscopy procedures;

FIGS. 9A-D illustrate a flexible sheath and an access device for use in a colposcopy or hysteroscopy procedure;

FIGS. 10A-B illustrate an exemplar cross-section for an access device for use in a colposcopy or hysteroscopy procedure;

FIG. 11 illustrates an exemplar cross-section for an access device for use in a colposcopy or hysteroscopy procedure;

FIG. 12 illustrates another exemplar cross-section for an access device for use in a colposcopy or hysteroscopy procedure;

FIG. 13 illustrates another exemplar cross-section for an access device for use in a colposcopy or hysteroscopy procedure;

FIGS. 14A-K illustrate another access device;

FIGS. 15A-C illustrate steps for performing a colposcopy using an access device as disclosed herein;

FIGS. 16A-C illustrate steps for performing a hysteroscopy using an access device as disclosed herein; and

FIGS. 17A-B illustrate obtaining a tissue sample.

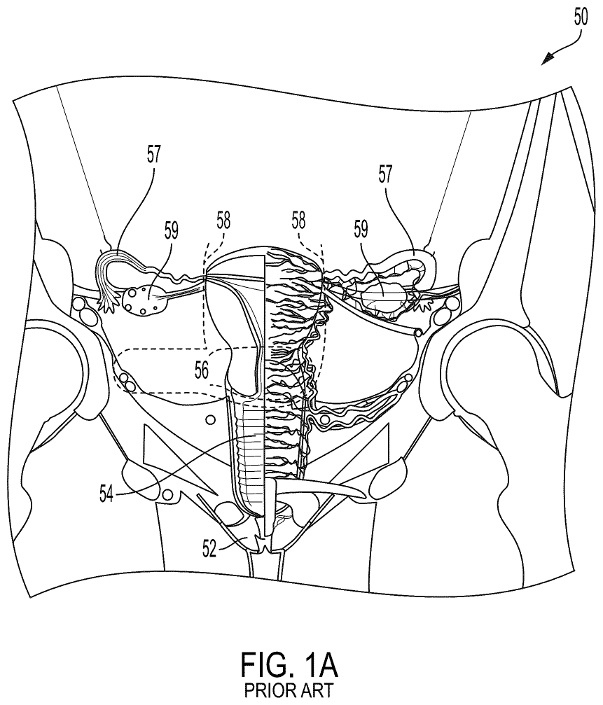

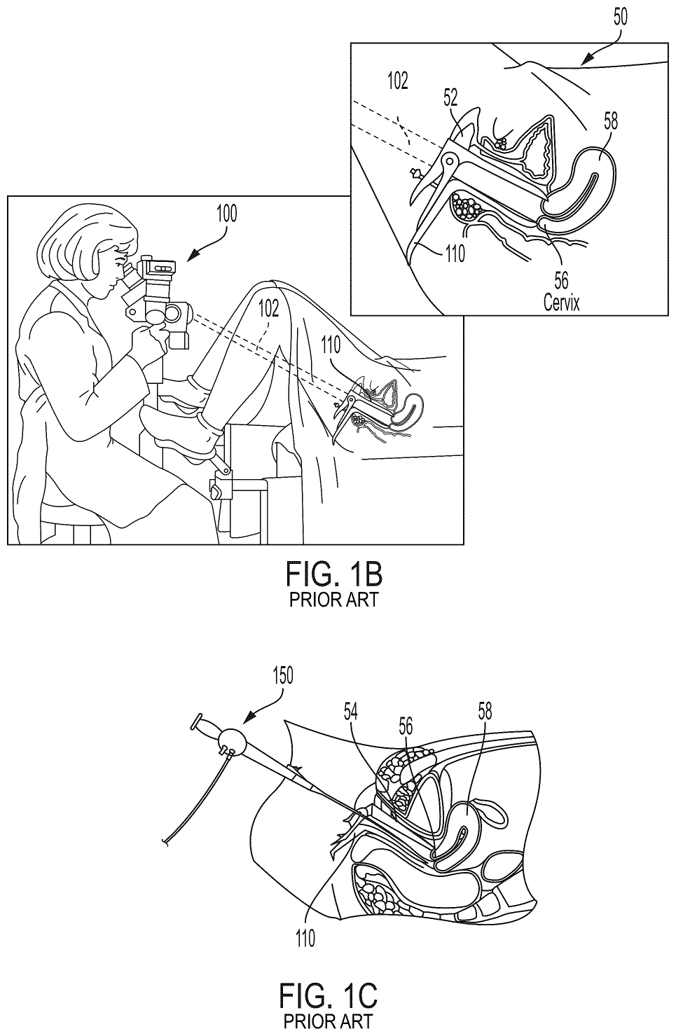

### DETAILED DESCRIPTION

### I. Devices

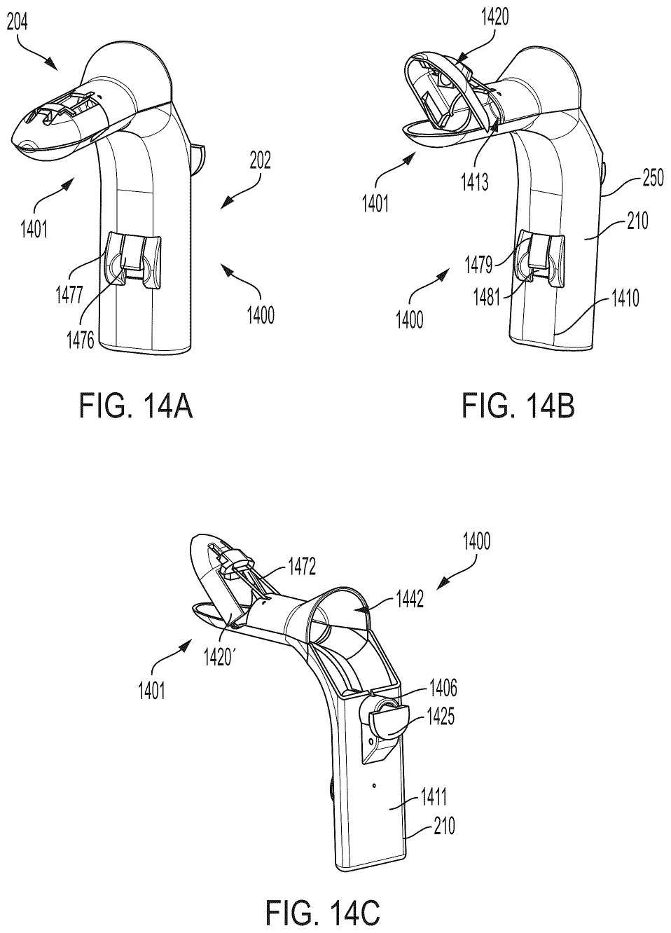

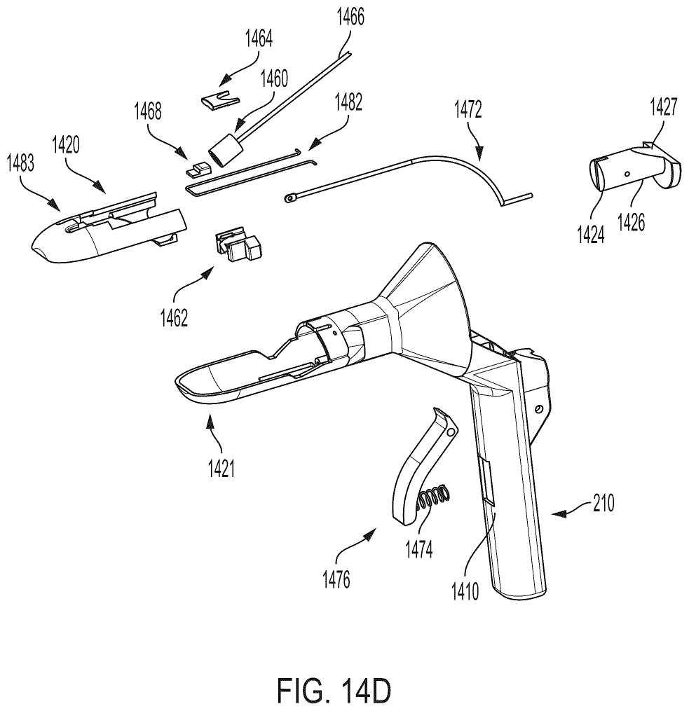

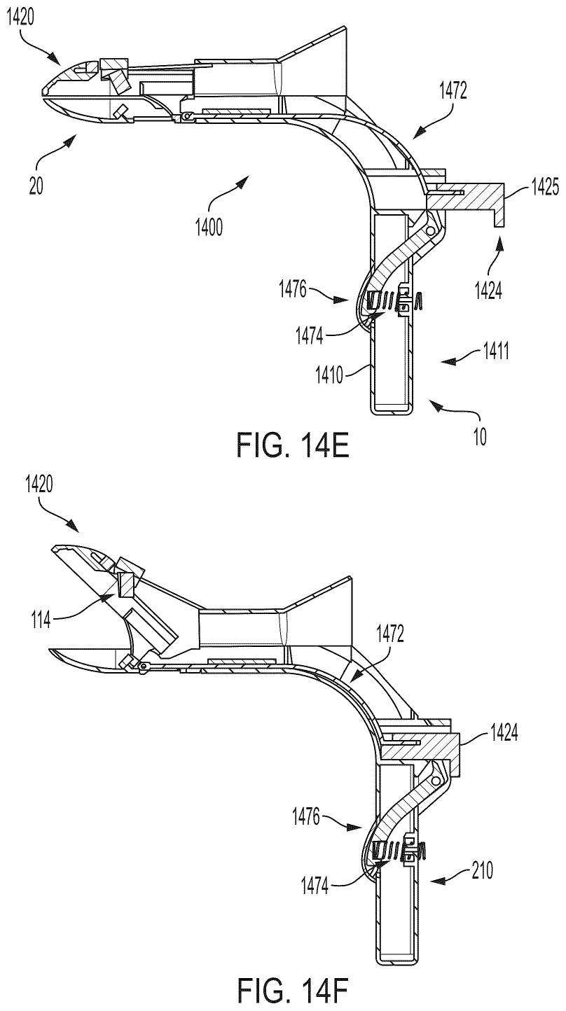

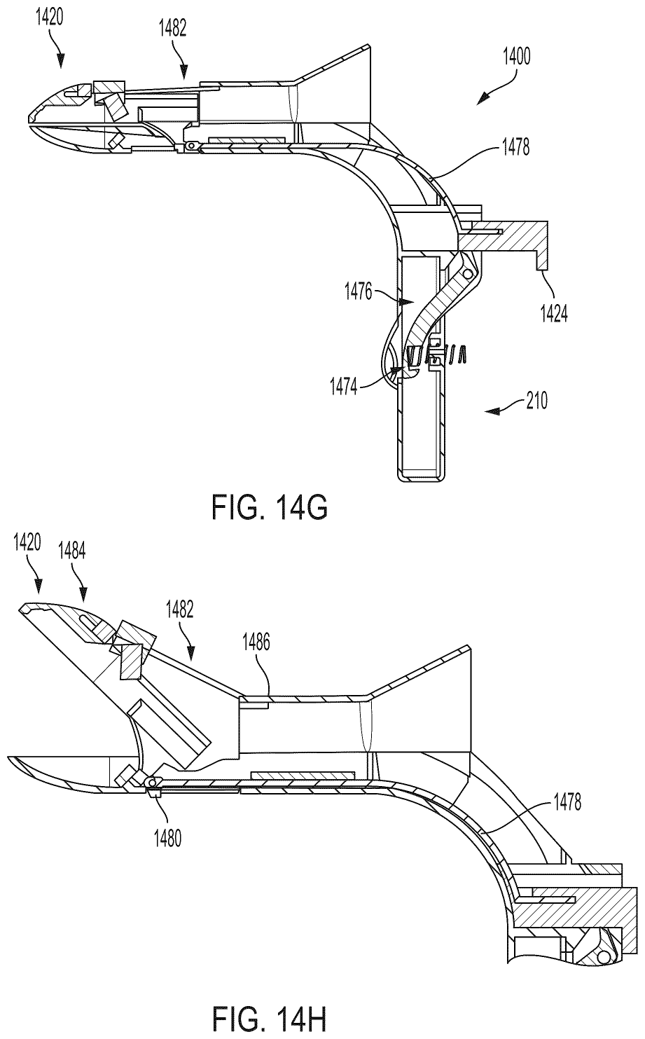

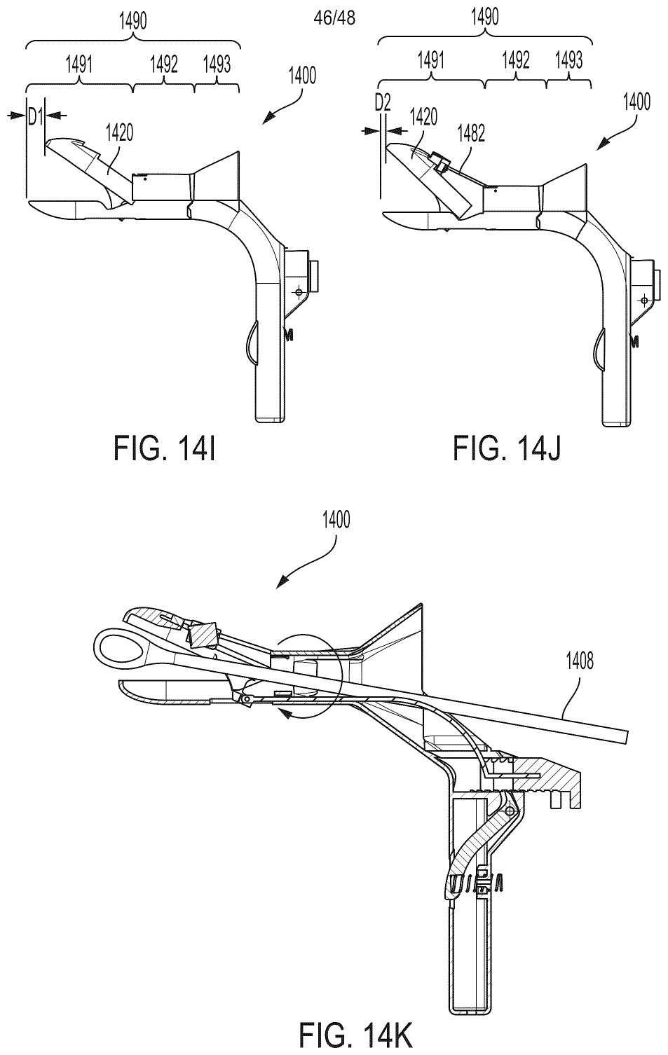

Access devices 200, 300, 400, 500, 600, 700, 900, 1000, 1400 are disclosed for lower genital tract access operable to, for example, obtain biological samples and for use in a colposcopy or hysteroscopy procedure. In general, the access devices 200, 300, 400, 500, 600, 700, 900, 1000,1400 have a first elongated member (such as first elongated member 202) and a second elongated member (such as second elongated member 204). An angle α from a first central axis, e.g. first elongated member central axis 11, through the first elongated member and to a second elongated member central axis, e.g. second elongated member central axis 21, through the second elongated member has a value of from 25 mm to 65 mm, more preferably 45 mm. The length 1 of the disclosed access devices from a proximal end 10 to a distal end 20 along the elongated member central axis 21 is from 125 mm to 200 mm, more preferably 130 mm to 175 mm, and even more preferably 138 mm. The length of a barrel portion of the access devices is from 60 mm to 100 mm (e.g., the portion distal to the conical aperture portion shown in FIG. 14), more preferably 87 mm. The distal aperture can have a diameter of from 15 mm to 30 mm, or have an oval shape with a diameter in a first axis of from 18 mm to 22 mm and a diameter in a second axis, perpendicular to the first axis, of from 26 mm to 30 mm. The height h of the access devices are from 140 mm to 220 mm, more preferably 160 mm to 200 mm, and even more preferably 185 mm. The open diameter of the distal end of the device can be from 40 mm to 60 mm, more preferably 48 mm. The handle portion has a length of from 90 mm to 130 mm, more preferably 111 mm, and a width of from 30 mm to 50 mm, more preferably 43 mm.

The widest external diameter, e.g. widest external diameter d1, of the barrel comprising the second elongated member 204 of the access devices 200 at its widest point is from 20 mm to 26 mm, more preferably 23 mm. The distal external diameter of the barrel of the access devices, e.g. distal external diameter d2, is from 29 mm to 37 mm, more preferably 33.5 mm. The first elongated member has a first elongated member proximal end (e.g., first elongated member proximal end 201) and a first elongated member distal end (e.g., first elongated member distal end 203). The first elongated member proximal end faces towards the second elongated member and the first elongated member distal end towards the user. The second elongated member (e.g. second elongated member 204) has a second elongated member proximal end (e.g. second elongated member proximal end 201′) and a second elongated member distal end (e.g., second elongated member distal end 203′).

In some configurations, the second elongated member can have a retractable sheath upper surface (e.g., retractable sheath upper surface 221) and a second elongated member upper surface (e.g., second elongated member upper surface 223). Additionally, the access devices can include, for example, one or more channels (e.g., channels 262), such as working channels. The one or more working channels can accommodate an image capture device, a light, and or tools. The one or more channels can allow passage of one or more of: medicinal fluids; fluids for cleaning the interior of the vaginal canal; fluids for cleaning the exterior of the cover; gas for inflating inner spaces of the body; surgical tools for collecting biological samples; cameras; lights; and sample collection devices.

At least one of the one or more channels can be a fluid channel. The fluid channel can be integrated into the camera module such that the fluid channel allows movement alongside the camera as an assembly. When the camera module is advanced through the cervix and into the uterus, fluid can be introduced through the fluid port to provide distention of the uterus to allow visualization. Where the access device is configured and operable for at home use a working channel is not required. A standard Luer type connector can be readily accessed via flexible tubing at the rear of the handle (not shown in the drawings).

The image capture device can be, for example, a camera and an associated optical lens or lens system. The image capture device is positionable within the access device housing so that the image capture device is operable a distal housing end of the housing and operable to collect one or more images of a visual field located outside the distal housing end of the housing. In some configurations, the image capture device and associated optical lens or lens system is removably positioned within the housing via an aperture in the housing at a proximal end of the second elongated member 204 and operable to collect one or more images of a visual field located outside the distal end of the second elongated member of the housing. The one or more optical lenses is chosen from the following group of lenses: a narrow field of view lens; a fish-eye lens; an omni-directional lens; and a lens having zoom-in and zoom-out functions. Additionally, the one or more image capture devices can be operable to acquire still images and/or and video images. The images can also be streamed via an electronic device. The acquired images can also be in the visible, near infrared, infrared, or ultraviolet spectral regions.

An illumination assembly can be provided, such as a light source (e.g., light emitting diodes). The illumination assembly is operable to illuminate a visual field located outside the distal end of the second elongated member of the housing. The illumination assembly can be removably positioned within the housing so that the illumination assembly is operable via an aperture at the distal end of the second elongated member of the housing. The illumination can be in the visible, near infrared, infrared, or ultraviolet spectral regions. The illumination assembly is operable to generate an illumination in an illumination spectral range substantially the same an image capture sensitivity spectral range.

In some configurations the light is built into the access device, in other configurations the light is operable through a working channel. In an embodiment the camera module with built in fluid port could be configured to reside outside the working channel to allow other instruments to be passed through the working channel under visualization of the camera. When the camera with fluid port is introduced into the uterus, visualization of the uterine cavity can be achieved.

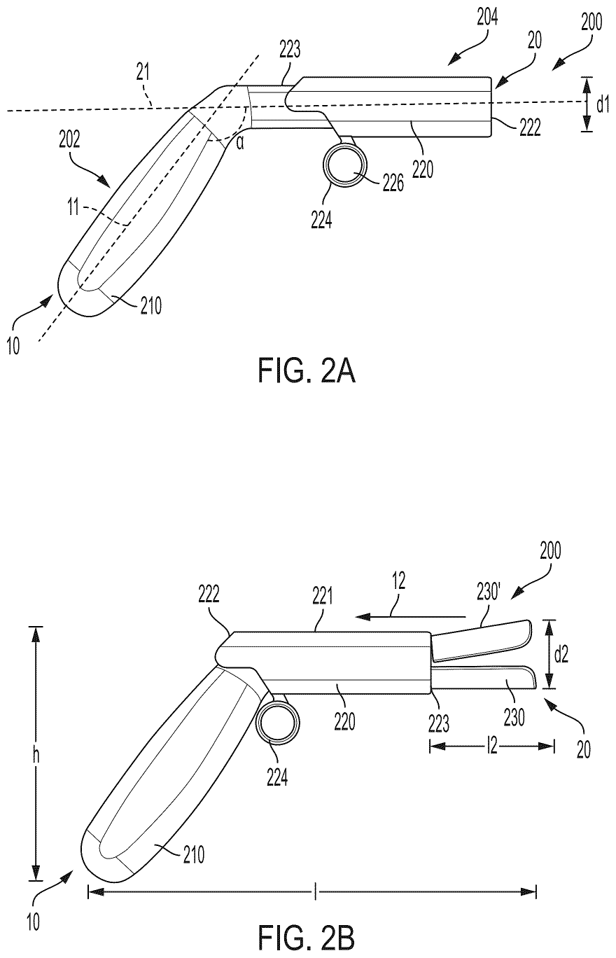

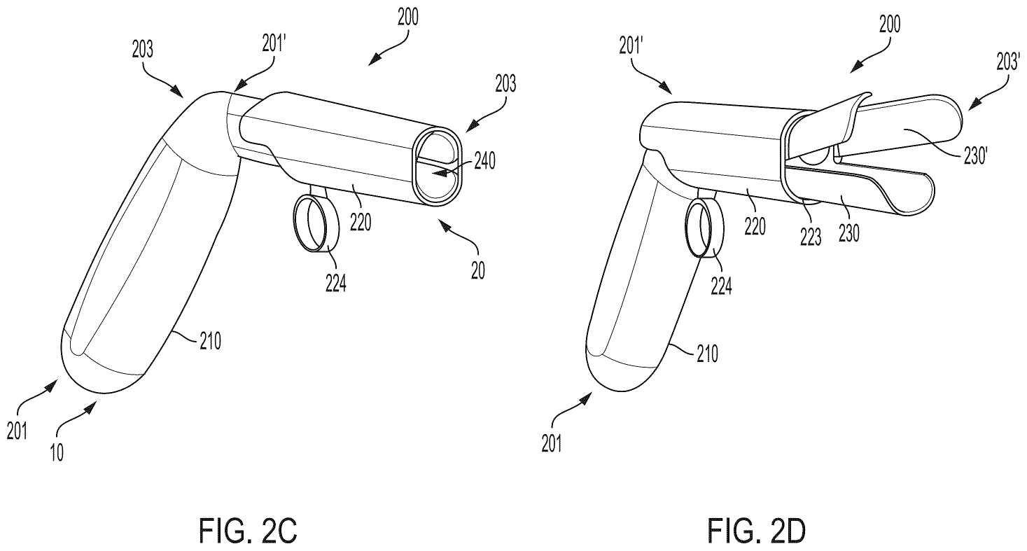

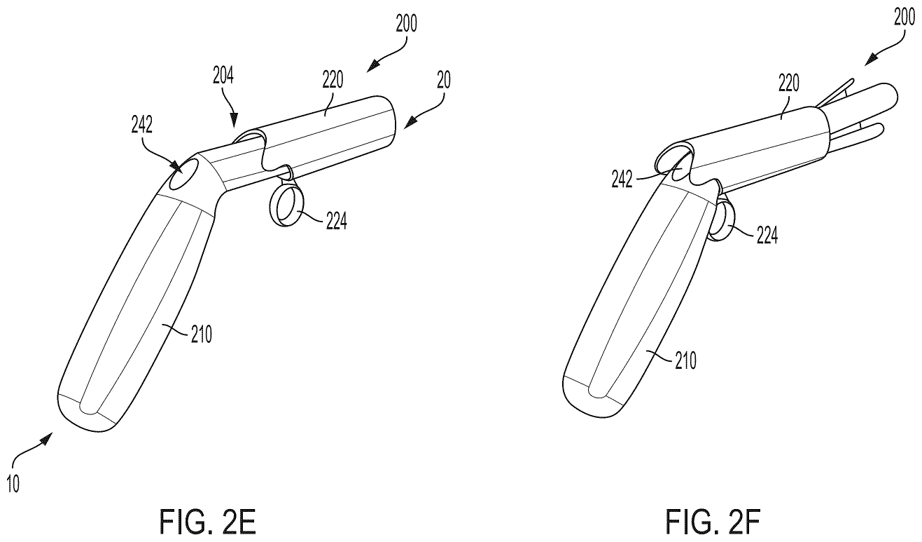

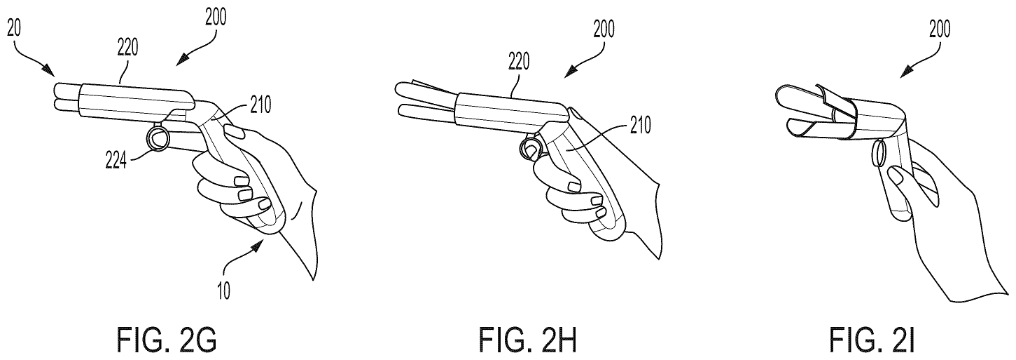

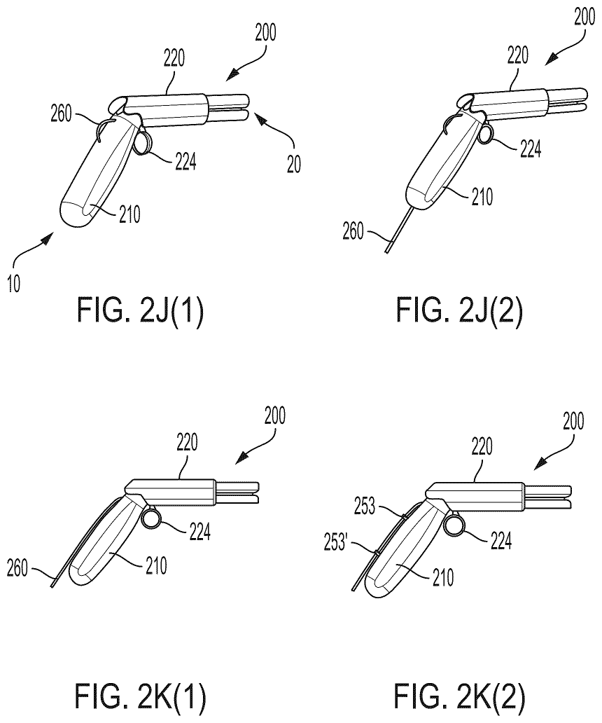

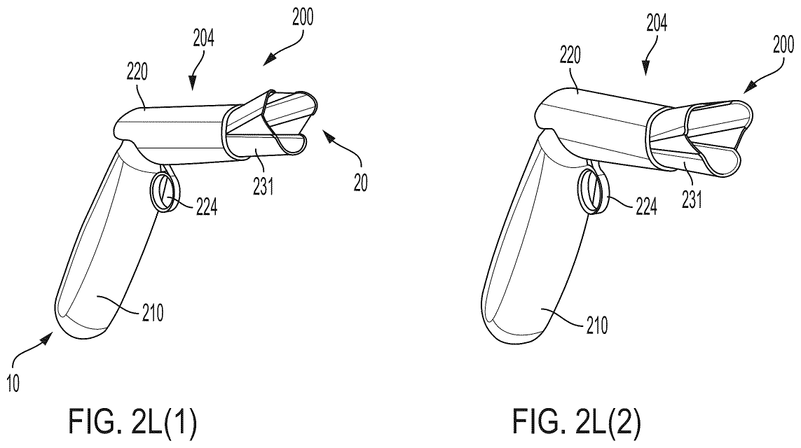

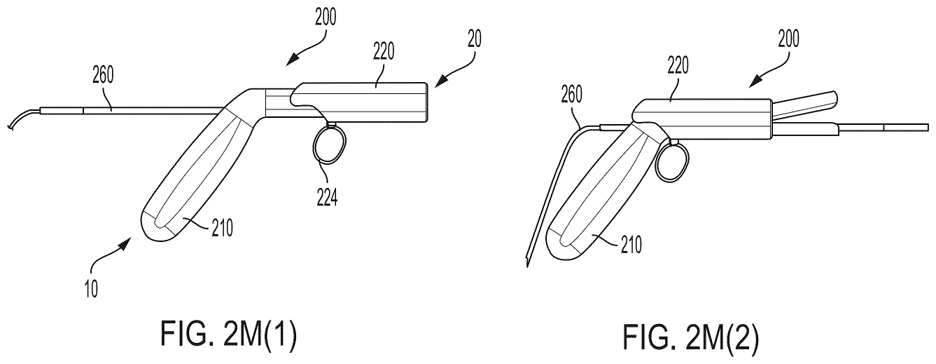

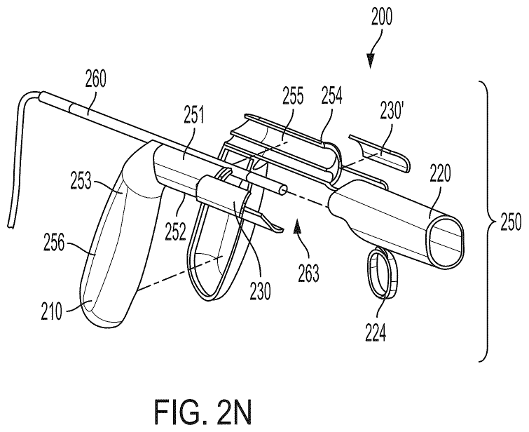

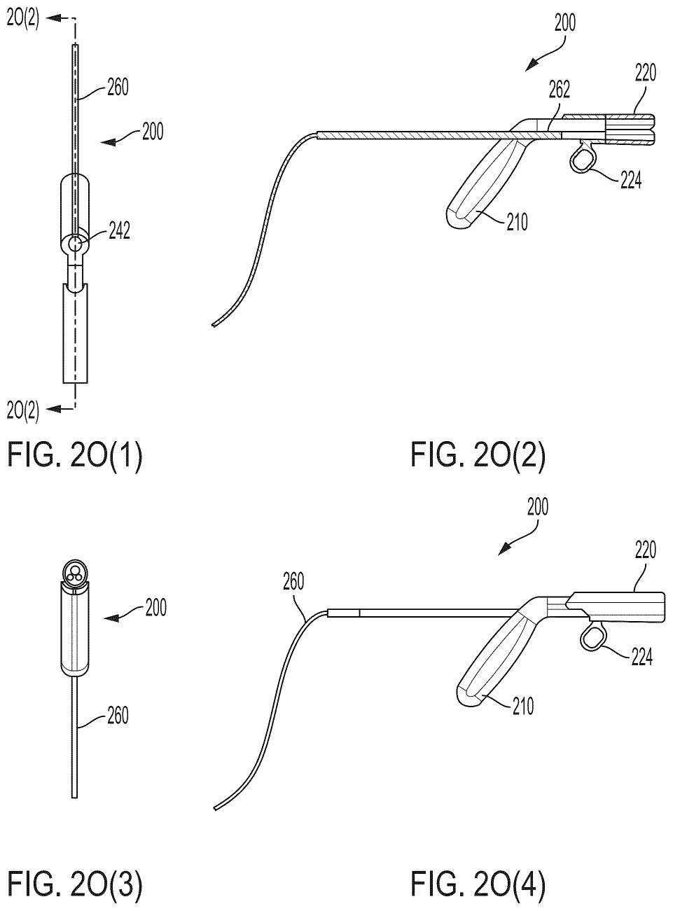

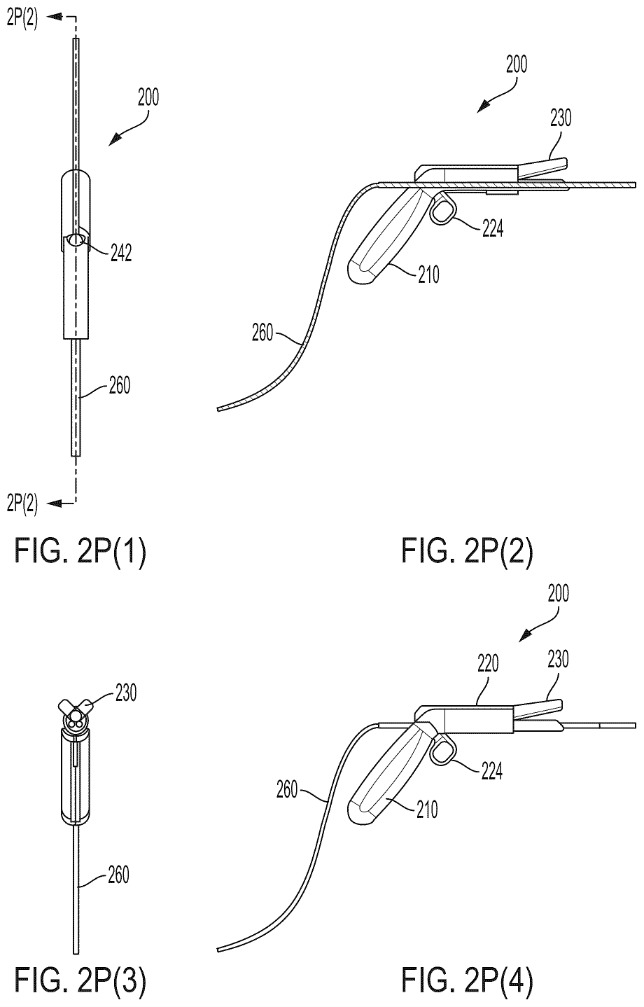

Turning now to FIGS. 2A-P an embodiment of an access device 200 for use in a colposcopy or hysteroscopy procedure is illustrated. In general, the access device 200 has a housing 250 with an upper housing surface 251, a lower housing surface 252, a first side surface 253, and a second side surface 254. The housing also has an interior housing surface 255 and an exterior housing surface 256. The housing 250 has a first elongated member 202 which can function as a handle 210 and a second elongated member 204 which is the barrel of the device. The first elongated member 202 and the second elongated member 204 can be integrally formed either partially or completely.

The access device 200 can have a smooth exterior surface, as shown, or include texturing or shaping on all or portions of the exterior surface to facilitate secure holding of the access device 200 during use. For example, a series of concave recesses (not shown) can be provided on the lower housing surface 252 of the first elongated member 202 wherein the concave recesses are shaped to receive a digit of a user's hand when the user grips the handle 210 of the access device 200.

FIG. 2A is a side view of the access device 200. The access device 200 has a proximal end 10 and a distal end 20. The first elongated member 202 of the proximal end 10 forms a handle 210 having a first elongated member central axis 11. The first elongated member 202 extends into a second elongated member 204. The distal end 20 of the access device 200 has a retractable sheath 220 positioned about the second elongated member 204 with a second elongated member central axis 21. The retractable sheath 220 has a retractable sheath proximal end 222 and a retractable sheath distal end 223 as illustrated. As will be appreciated by those skilled in the art, the retractable sheath proximal end 222 can be curved (as illustrated), slanted or blunt without departing from the scope of the disclosure. As will be appreciated by those skilled in the art, the retractable sheath distal end 223 can be blunt (as illustrated), slanted or curved without departing from the scope of the disclosure.

A distal end diameter controller 224 is provided on the retractable sheath 220 that is operable to retract the retractable sheath 220 in a proximal direction 12 during use to provide a distal end diameter control. The distal end diameter controller 224 has controller surface and can be circular with a distal end diameter controller aperture 226 sized to receive a finger therethrough, similar to a trigger guard. Additionally, the distal end diameter controller 224 can be positioned on the retractable sheath lower surface 221′ of the retractable sheath 220 and toward the proximal end 10 of the retractable sheath 220 as illustrated. The retractable sheath 220 can have a first length along the upper surface and a second length, shorter than the first length, along the lower surface. An angled proximal surface can be provided that allows the retractable sheath 220 to surround a distal portion of the handle 210 or be positioned adjacent the handle 210 on the distal end of the first elongated member 202 when the retractable sheath 220 is moved in a proximal direction while the retractable sheath upper surface 221 is positioned along an interior facing proximal surface of the handle 210. The widest external diameter, e.g. widest external diameter d1, of the barrel of the retractable sheath 220 at its widest point is from 20 mm to 26 mm, more preferably 23 mm.

As shown in FIG. 2B, when the retractable sheath 220 is moved proximally, two or more curved members 230, 230′ are exposed at the distal end and extend away from the second elongated member central axis 21. When the retractable sheath 220 is retracted, the length 12 of the exposed two or more curved members is from 30 mm to 40 mm, more preferably 35 mm. The external diameter of the curved members 230, 230′ of the access devices, e.g. distal external diameter d2, increases to from 29 mm to 37 mm, more preferably 33.5 mm.

FIGS. 2C and 2D is a perspective view of an access device 200 from the distal end 20 of the access device 200. The distal end of the access device 200 is an expandable distal end 244. The second elongated member 204 and retractable sheath 220 define a distal aperture 240. From the distal end 20 of the access device 200 the working channel 262 and light are operable. As the retractable sheath 220 moves proximally, the curved members 230, 230′ (in this configuration three curved members) extend away from the second elongated member central axis 21. FIG. 2C shows the access device 200 prior to retracting the retractable sheath 220 and FIG. 2D shows the access device 200 after retracting the retractable sheath 220.

FIGS. 2E and 2F is a perspective view of an access device 200 from the proximal end 10 of the access device 200. The second elongated member 204 has a proximal housing aperture 242 in communication with the distal aperture 240 shown in FIG. 2C. The proximal housing aperture 242 provides access to the working channel formed within the second elongated member 204. FIG. 2E shows the access device 200 prior to retracting the retractable sheath 220 and FIG. 2F shows the access device 200 after retracting the retractable sheath 220. As shown in FIG. 2F, the distal end of the retractable sheath 220 extends past the outer surface of the first elongated member 202.

FIGS. 2G-H are side views of the access device 200 in use. In FIG. 2G, a user has an index finger positioned in a distal end diameter controller aperture 226 and the remaining fingers and thumb are shown gripping the handle 210. In FIG. 2H, the user has drawn the index finger positioned in the distal end diameter controller aperture 226 proximally toward the handle 210 causing the retractable sheath 220 to be drawn proximally away from the distal end of the access device 200, thereby exposing the two or more curved members 230, 230′. Once the two or more curved members are released from within the retractable sheath 220, the two or more curved members expand away from the second elongated member central axis 21 resulting in a larger distal diameter for the access device 200. FIG. 2I is a perspective view of an access device 200 with the distal end diameter controller 224 drawn in a proximal most position and the two or more curved members 230, 230′ achieving a maximum distal diameter. FIG. 2I illustrates a configuration where the two or more curved members is three members. The curved members can have the same length along the diameter or different lengths.

FIGS. 2J(1) and 2J(2) illustrate an embodiment of an access device 200. A camera cable 260 is shown extending from the backside of the handle 210. The camera cable 260 could also be positioned within an access device cavity 263 within the housing 250 or a dedicated working channel 262 within the second elongated member 204. A port can be provided with a Luer fitting (not shown) that enables an HCP can introduce saline through a syringe or pump during use to distend the uterus when the camera module assembly is introduced into the uterus as in hysteropic procedures. One or more cable anchors 246, 246′ can be provided to secure the cable to an exterior surface of the handle 210.

FIGS. 2K(1) and 2K(2) illustrate another embodiment of an access device 200. The difference between the configuration in FIG. 2J and FIG. 2K is that the camera cable 260 is routed through an interior portion of the handle 210 in the configuration shown in FIG. 2J while the camera cable 260 is routed outside the handle 210 in the configuration illustrated in FIG. 2K.

FIGS. 2L(1) and 2L(2) illustrate another embodiment of an access device 200 from a distal end 20 perspective. When the retractable sheath 220 is pulled proximally toward the handle 210, instead of exposing the two or more curved members 230, 230′ as shown in FIG. 2B, the exposed expandable member 231 is a single piece which unfurls or unfolds from a constricted configuration that is sized to fit within the retractable sheath 220 to an unconstructed configuration with a diameter at the distal end that is greater than the diameter of the distal end of the second elongated member 204.

FIGS. 2M(1) and 2M(2) illustrate another embodiment of an access device 200 from a side view. When the retractable sheath 220 is pulled proximally, instead of exposing the two or more curved members 230, 230′ that extend away from the second elongated member central axis 21 as shown in FIG. 2B, a first curved member 230 extends away from the second elongated member central axis 21 while the second curved member 231′ does not. A camera cable 260 is shown extending from the distal end of the handle 210. The camera module/fluid port assembly could be a slideable assembly positioned within a hand piece or handle 210. Moreover, the camera module/fluid port can be configured to reside within a dedicated channel as opposed to being positioned in a working channel. Thus, the access device 200 can form two channels within the interior of the second elongated member 204. This configuration allows, for example, a colposcopy procedure such as cervical sampling and cervical biopsy to be performed as the camera and working channel are both present (versus running the camera module assembly through the working channel).

FIG. 2N is an exploded view of an access device 200 and FIGS. 2O-P illustrate multiple views in a closed or open configuration. As described above, the access device 200 has a housing 250 with an upper housing surface 251, a lower housing surface 252, a first side surface 253, and a second side surface 254. As illustrated, the housing 250 also has an interior housing surface 255, and an exterior housing surface 256. The housing 250 has a first elongated member 202 which can function as a handle 210 and a second elongated member 204. The first elongated member 202 and the second elongated member 204 can be integrally formed either partially or completely.

FIG. 2O(1)-(4) are upper, lower, cross-sectional and side views of an access device 200 prior to retracting the retractable sheath.

FIG. 2P(1)-(4) are upper, lower, cross-sectional and side views of an access device 200 after retracting the retractable sheath.

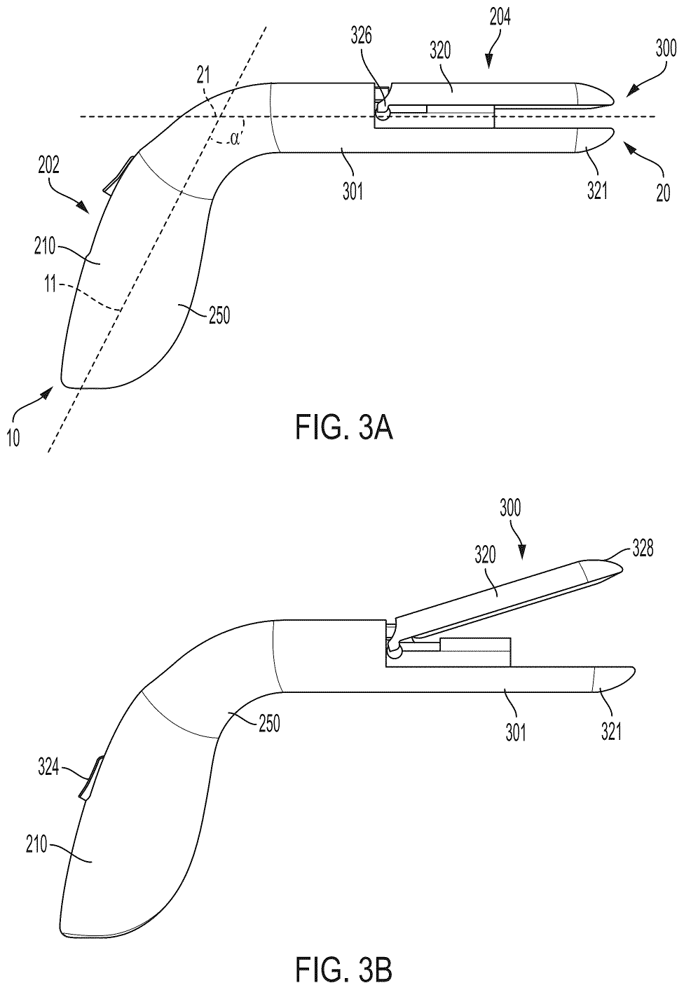

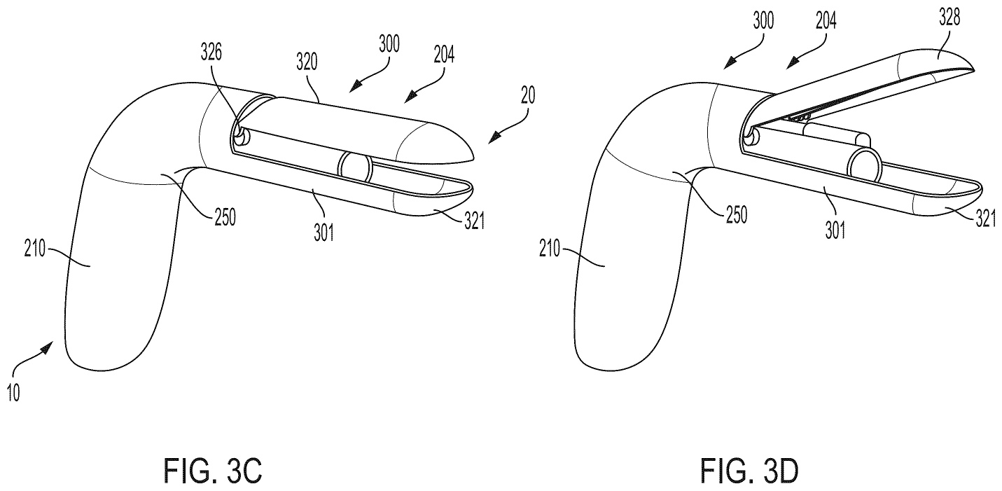

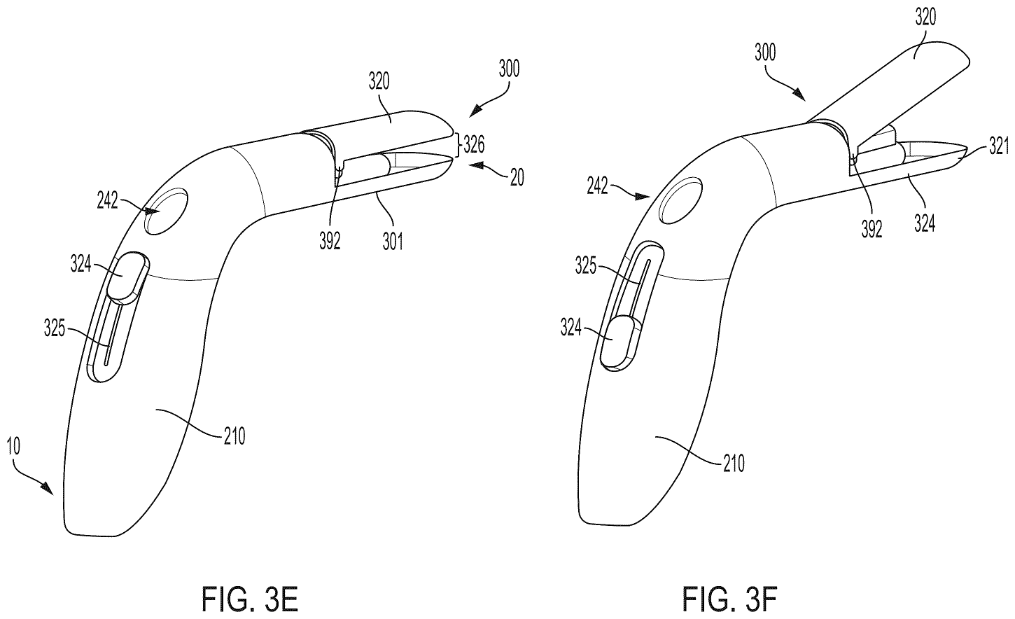

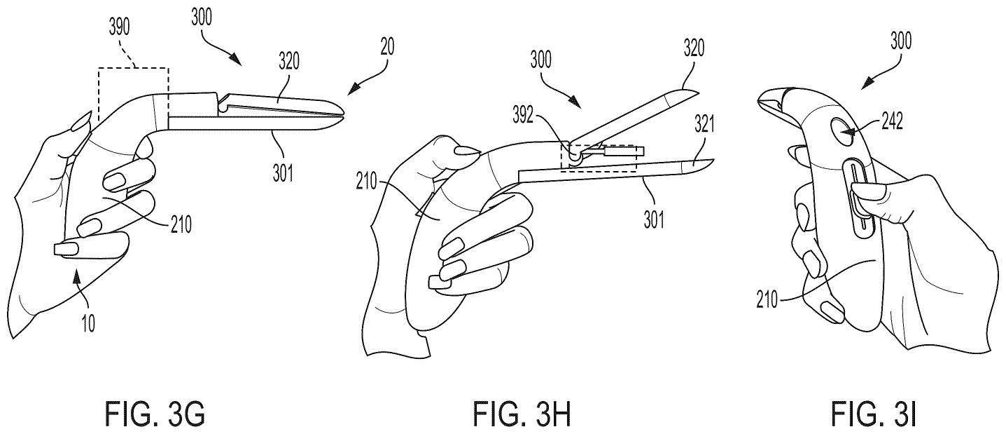

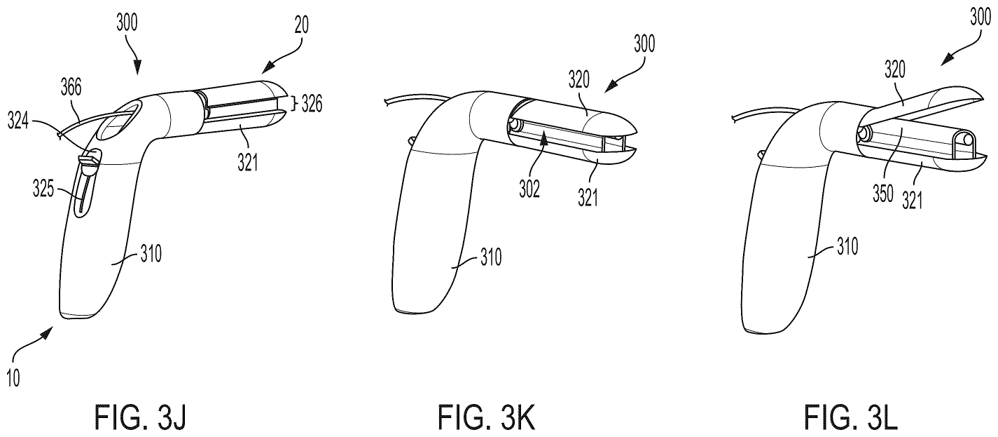

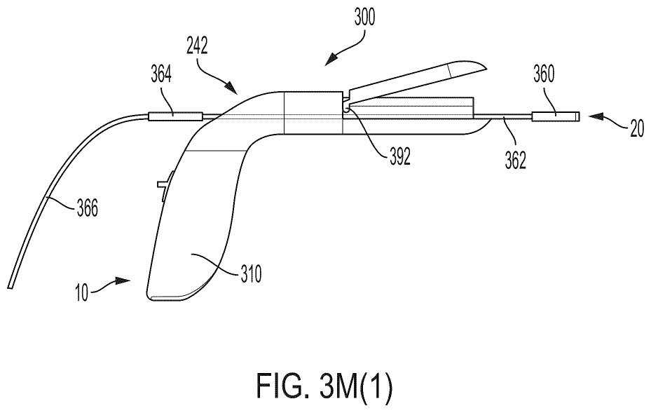

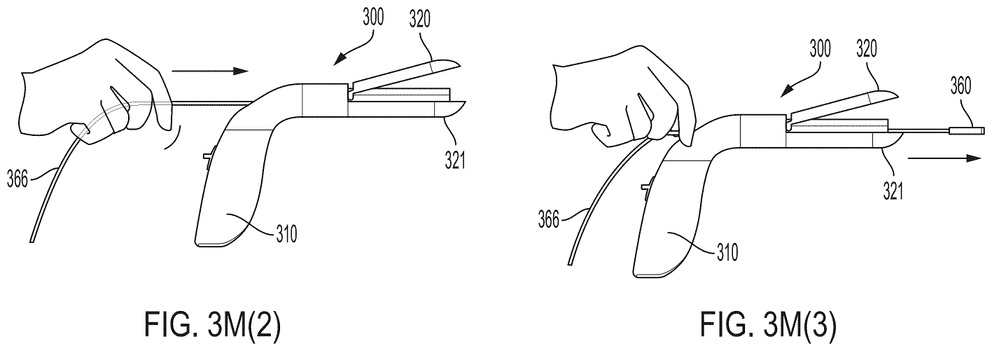

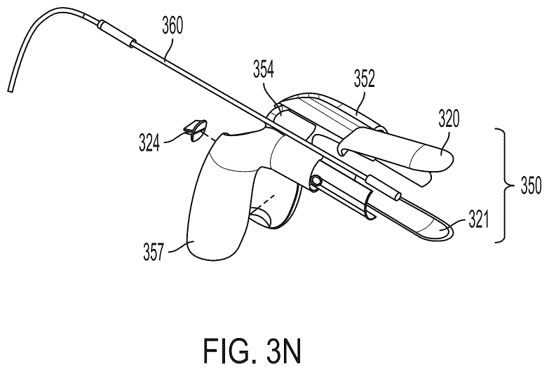

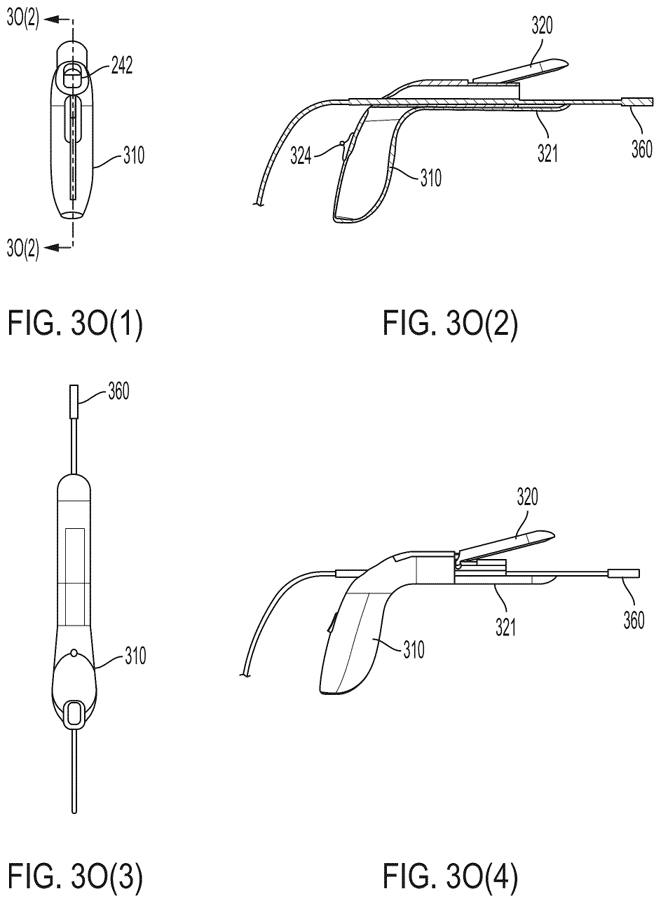

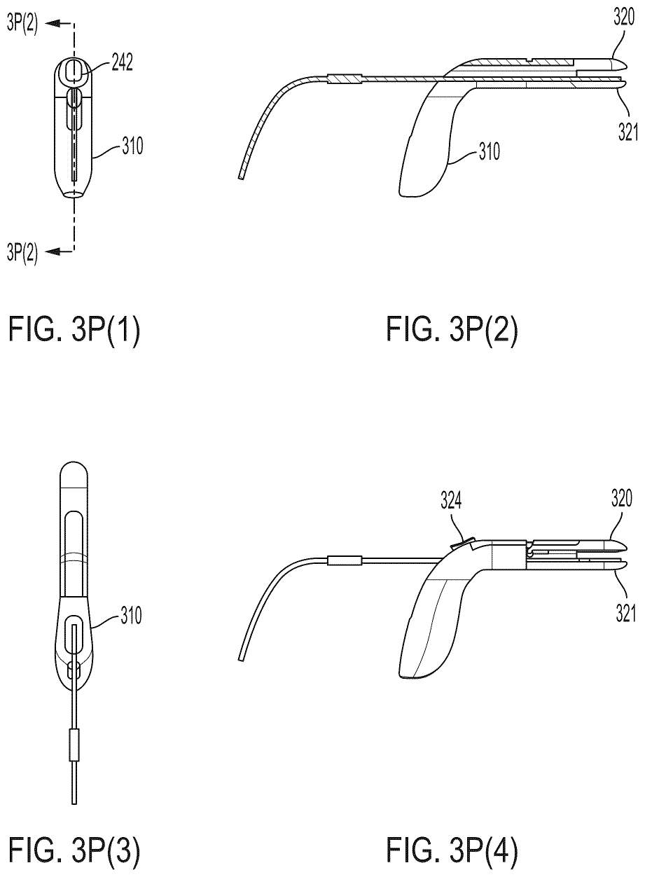

FIGS. 3A-P illustrate another embodiment of an access device 300 for use in a colposcopy or hysteroscopy procedure. As shown in FIGS. 3A-B, the first elongated member 202 of the proximal end 10 forms a handle 210 having a first elongated member central axis 11. The second elongated member 204 has a rotatable member 320. The rotatable member 320 is part of the second elongated member and is positioned distal to the handle 210. The lower portion 301 of the second elongated member 204 can be formed integrally with the housing 250 of the access device 300 and form a non-rotatable member 321. Either or both of the rotatable member 320 and the non-rotatable member 321 can have a curved distal end 328 as illustrated (curved or rounded in a side profile). Additionally, an elongated gap 326 can be present between the rotatable member 320 and the non-rotatable member 321. The elongated gap 326 can extend along the second elongated member central axis 21.a gap can be present between the bottom of the second elongated member 204 and the rotatable member 320 forming the top of the second elongated member 204. Either or both of the rotatable member 320 and the non-rotatable member 321 can have a curved distal end 328 as illustrated.

A distal end diameter controller 324 is provided on the handle 210 that is operable to rotate the rotatable member 320 at a hinge 327 away from the second elongated member central axis 21. A distal end diameter controller 324 is provided on the handle 210 can be a button or slider (e.g., slidable button) positioned on the handle 210.

FIGS. 3C-D illustrate the access device 300 from a partial distal perspective view. Although not shown, the rotatable member 320 can engage the body of the access device 300 at a pivot point 392 via a detent—recess configuration. The detent can be positioned on the access device body while the recess is positioned on an interior facing surface of the rotatable member 320 or vice versa. The use of the detent-recess to secure the rotatable member 320 to the body of the access device 300 allows rotation of the rotatable member 320 about a rotation axis perpendicular to the second elongated member central axis 21 in a direction away from the second elongated member central axis 21. When the rotatable member 320 is positioned closest to the lower member the access device 300 is in a closed configuration. When the rotatable member 320 is rotated away from the second elongated member central axis 21, the access device is in an open configuration. The distal end 20 has a curved surface. When the access device 300 is in a closed configuration, the curved distal surface facilitates comfortable insertion of the access device 300 into the vagina.

As shown in FIGS. 3E-F, the distal end diameter controller 324 can be a button that slides within a slot 325 or channel on the handle 210 to control rotation of the rotatable member 320 away from the second elongated member central axis 21. The second elongated member 204 has a proximal housing aperture 242. The proximal housing aperture 242 provides access to the working channel within the second elongated member 204. Moving the distal end diameter controller 324 within the slot 325 moves the rotatable member 320 from a closed configuration (FIG. 3E) to an open configuration (FIG. 3F).

FIGS. 3G-I are side views of the access device 300 in use. In FIG. 3G, a user has a thumb positioned on a distal end diameter controller 324 and the remaining fingers are shown gripping the handle 210. As shown, a working channel length 390 is shown from about the proximal housing aperture 242 and terminating prior to the pivot point for the rotatable member 320. In some configurations, the working channel length and is selected to allow tools to pass therethrough while also ensuring that the distal action of the tool is not impeded due to the length of the channel. In FIG. 3H, the user has moved the distal end diameter controller 324 causing the rotatable member 320 to translate away from the second elongated member central axis 21. FIG. 3I is a rear perspective view of an access device 300 with the distal end diameter controller 324.

FIGS. 3J-L illustrate another embodiment of an access device 300 from a distal perspective. When the distal end diameter controller 324 is moved from a first position to a second position, the rotatable member 320 moved from a closed configuration (FIG. 3K) to an open configuration (3L). Positioned within a space 302 between the non-rotatable member 321 and the rotatable member 320. In this configuration the housing 250 defines an interior cavity within the interior of the non-rotatable member 321 and the rotatable member 320. The interior of the housing 250 is sized to receive, for example, a tool.

FIGS. 3M(1)-M(3) illustrate the access device 300 in use from a side view. As shown in FIG. 3M(1) an image capture device such as an endoscope camera 360 is positioned at a distal end of a semi-rigid cable 362. The semi-rigid cable 362 passes through a working channel within the access device 300. The proximal end of the semi-rigid cable 362 through the proximal housing aperture 242. An endoscope camera handle 364 is provided on the semi-rigid cable 362 at a location external to the access device 300 at the proximal end. Proximal to the endoscope camera handle 364 is a flexible cable 366. As shown in FIG. 3M(2), the endoscope camera handle 364 allows the user to maneuver the endoscope camera 360 from outside the access device 300. As shown in FIG. 3M(3), the endoscope camera 360 can be advanced distally as far as needed to achieve the desired level of visibility within the anatomy.

FIG. 3N is an exploded view of an access device 300 with the endoscope camera 360. The housing 350 of the access device 300 separates into a first half 351 and a second half 352. When the two halves of the housing 250 are assembled a housing cavity 354 is formed therein through which, for example, an endoscopic camera 360 can pass. The configuration of FIG. 3 is shown with a slider as the distal end diameter controller. However, a similar construction of the device body could be employed with other configurations or controllers without departing from the scope of the disclosure.

FIGS. 3O(1)-3O(4) and FIG. 3P(1)-3P(4) illustrate multiple views in a closed or open configuration. As shown in FIG. 3O, the distal end diameter controller 324 is positioned below the proximal housing aperture 242 on the handle 210. As shown in FIG. 3P, the distal end diameter controller 324 is positioned above the proximal housing aperture 242.

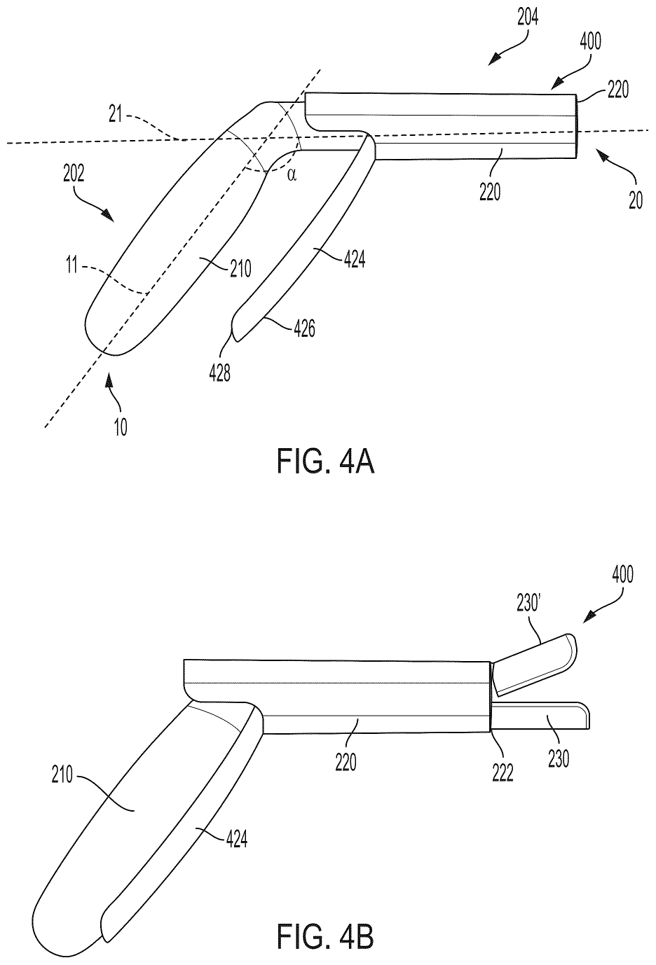

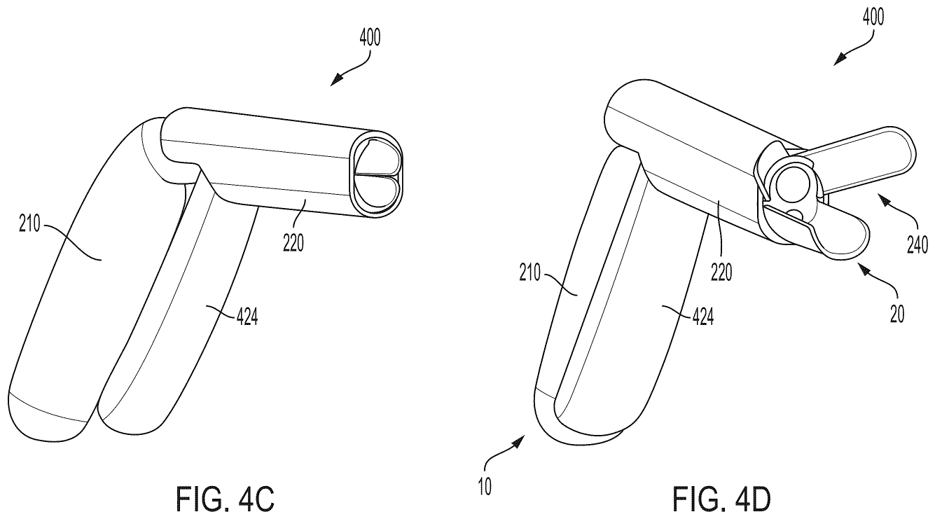

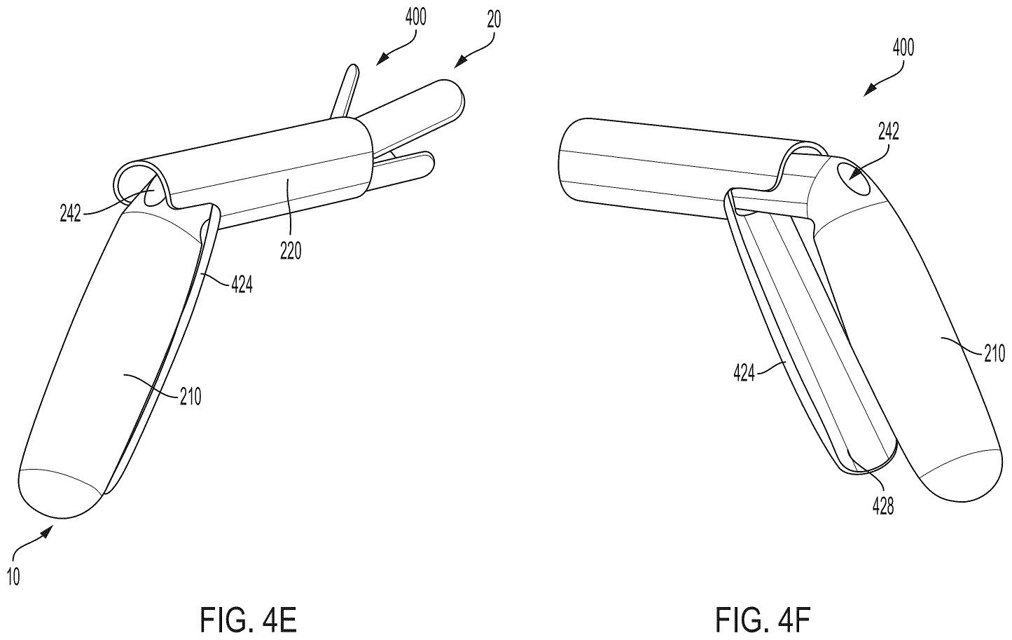

FIGS. 4A-F illustrate another embodiment of an access device 400 for use in a colposcopy or hysteroscopy procedure. The access device 400 has a proximal end 10 and a distal end 20.

FIG. 4A is a side view of the access device 400. The first elongated member 202 of the proximal end 10 forms a handle 210 having a first elongated member central axis 11. The distal end 20 of the access device 200 has a retractable sheath 220 positioned about the second elongated member 204 with a second elongated member central axis 21. The retractable sheath 220 provided similar to the retractable sheath in FIG. 2. The retractable sheath proximal end 222 can be blunt. As will be appreciated by those skilled in the art, the proximal end can also be slanted or curved without departing from the scope of the disclosure. Similarly, the retractable sheath distal end 223 can be blunt, slanted, or curved.

A lever controller 424 is provided in communication with the retractable sheath 220 that is operable to retract the retractable sheath 220 in a proximal direction during use. The lever controller 424 can be an elongated member that rigidly forms part of the retractable sheath 220 and extends away from the retractable sheath 220 at an angle α that aligns with the handle 210. The angle and curvature of the lever controller 424 allows the lever controller 424 to engage the handle 210 when the lever controller 424 is moved in a proximal direction towards the handle 210. Additionally, the lever controller 424 can be positioned on the lower surface of the retractable sheath 220 and toward the proximal end 10 of the retractable sheath 220 as illustrated. Similar to the configuration in FIG. 2, the retractable sheath 220 can have a first length along the upper surface and a second length, shorter than the first length, along the lower surface. An angled distal surface allows the retractable sheath 220 to engage the handle 210 or be positioned adjacent the handle 210 on the proximal end when the retractable sheath is pulled proximally while the upper side of the retractable sheath 220 is positioned along the proximal surface of the handle 210.

As shown in FIG. 4B, when the retractable sheath 220 is pulled proximally, two or more curved members 230, 230′ are exposed. Additionally, the lever controller 424 can have a first convex surface 426 for engaging the user's hand and an opposing concave surface 428, or suitable handle conforming surface, for engaging the exterior surface of the handle 210 when the lever controller 424 is urged towards the handle 210.

FIGS. 4C and 4D is a perspective view of an access device 400 from the distal end 20 of the access device 400. The second elongated member 204 and retractable sheath 220 have a distal aperture 240. From the distal end 20 of the access device 200 the working channel and light are operable.

FIGS. 4E and 4F is a perspective view of an access device 400 from the proximal end 10 of the access device 400. The second elongated member 204 has a proximal housing aperture 242. The proximal housing aperture 242 provides access to one or more working channels within the second elongated member 204.

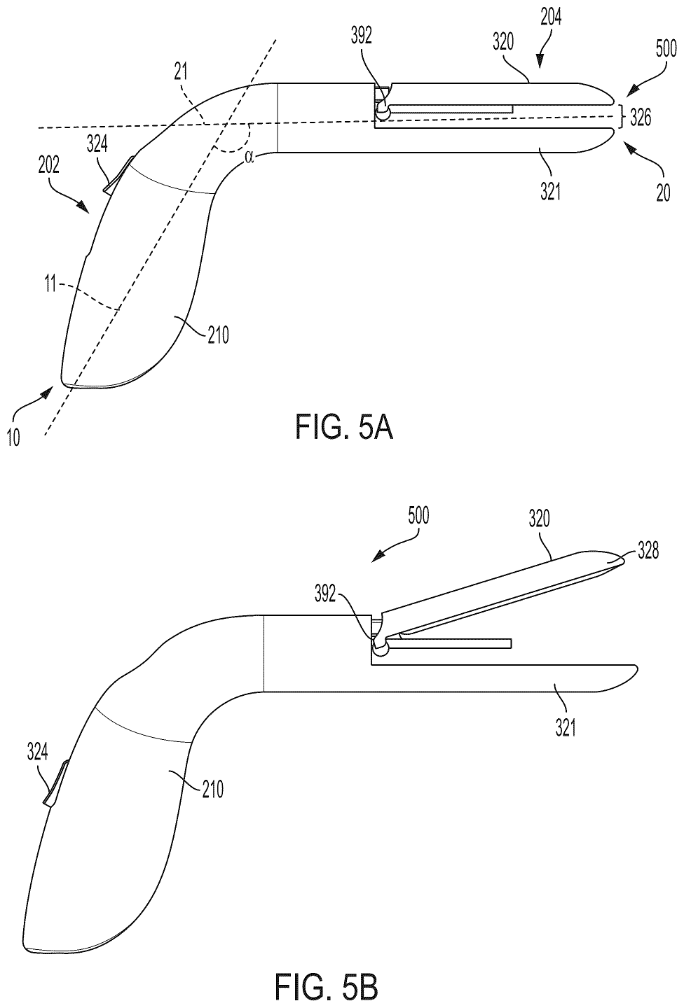

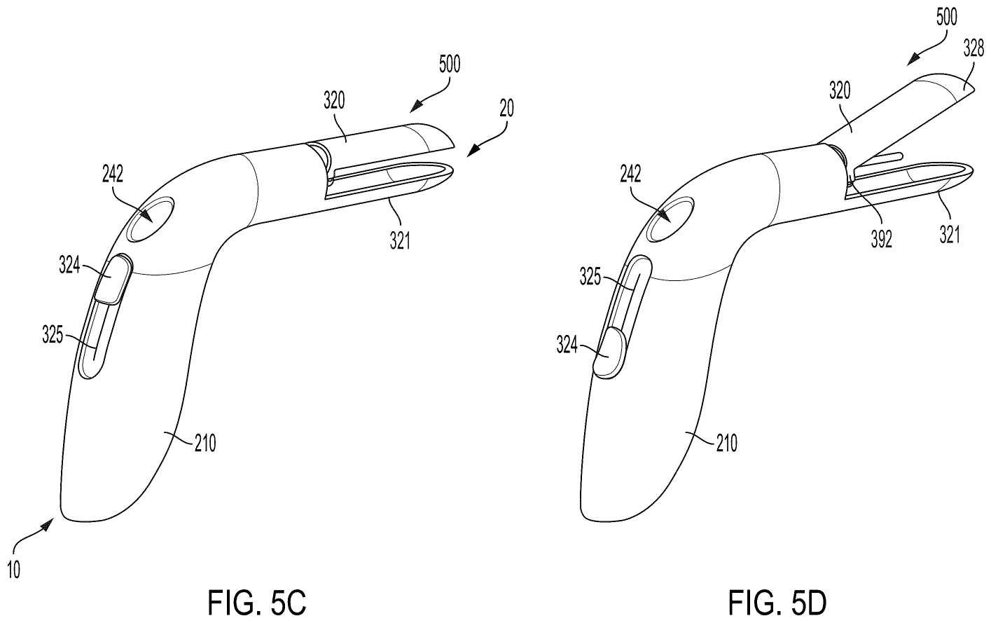

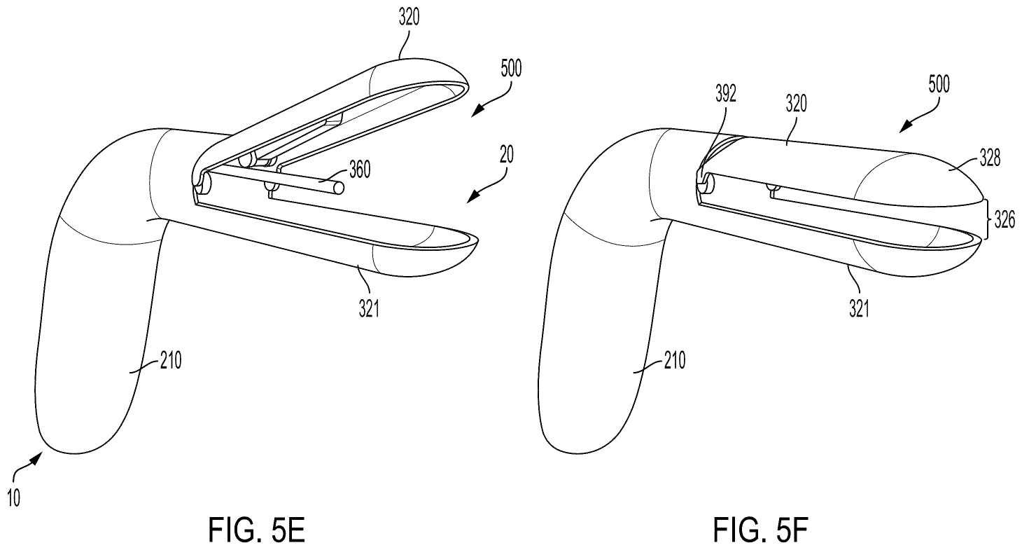

FIGS. 5A-F illustrate another embodiment of an access device 500 for use in a colposcopy or hysteroscopy procedure. The access device 500 has a proximal end 10 and a distal end 20.

As shown in FIGS. 5A-B, the first elongated member 202 of the proximal end 10 forms a handle 210 having a first elongated member central axis 11. The second elongated member 204 has a rotatable member 320. The rotatable member 320 is positioned distal to the handle 210 and forms part of the second elongated member 204. The rotatable member 320 can have a curved distal end 328 as illustrated. A distal end diameter controller 324 is provided on the handle 210 that is operable to rotate the rotatable member 320 at a hinge 327 away from a lower second elongated member section, non-rotatable member 321 and the second elongated member central axis 21. When the rotatable member 320 is in a closed position (e.g., before rotation of the rotatable member 320 away from the lower second elongated member section), an elongated gap 326 can be present between the rotatable member 320 and the non-rotatable member 321. The elongated gap 326 can extend along the second elongated member central axis 21.

FIGS. 5C-D illustrate the access device 500 from a partial distal perspective. Similar to FIG. 3, the distal end diameter controller 324 can be a button or a slider positioned on the handle 210. The rotatable member 320 can have an inward facing detent that engages a recess to secure the rotatable member 320 to the body of the access device 500 while allowing rotation of the rotatable member 320 away from the second elongated member central axis 21. When the rotatable member 320 is positioned closest to the lower member the access device 500 is in a closed configuration. When the rotatable member 320 is rotated away from the second elongated member central axis 21, the access device is in an open configuration. The distal end 20 has a curved surface. When the access device 300 is in a closed configuration, the curved distal surface facilitates insertion of the access device 300 into the vagina.

FIGS. 5E-F illustrate an access device 500 in an open configuration (FIG. 5E) and a closed configuration (FIG. 5F). In FIG. 5E the distal end of a tool, such as a camera 360 is visible.

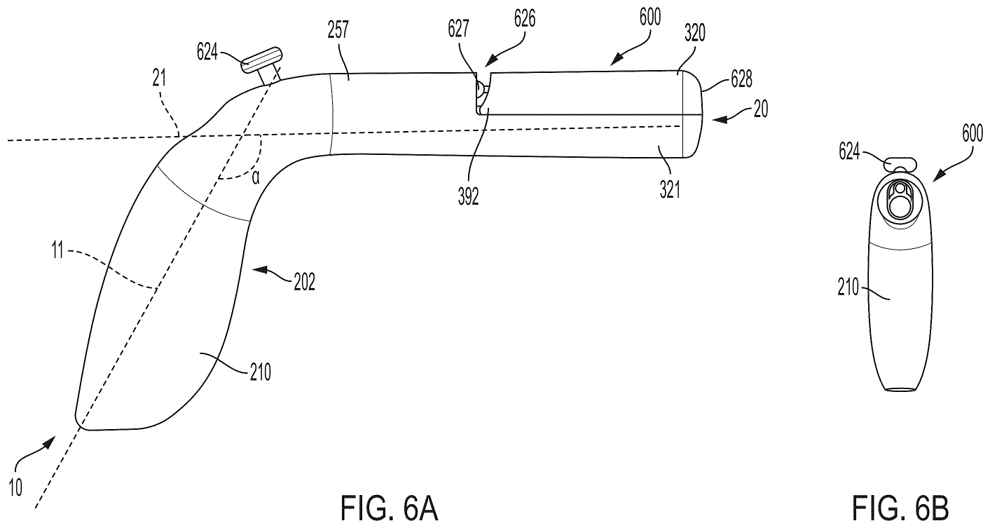

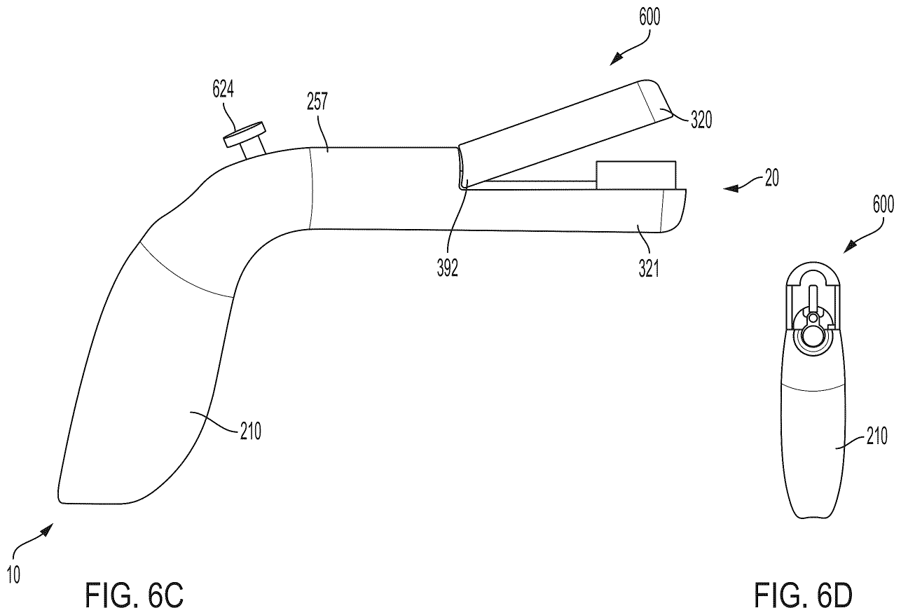

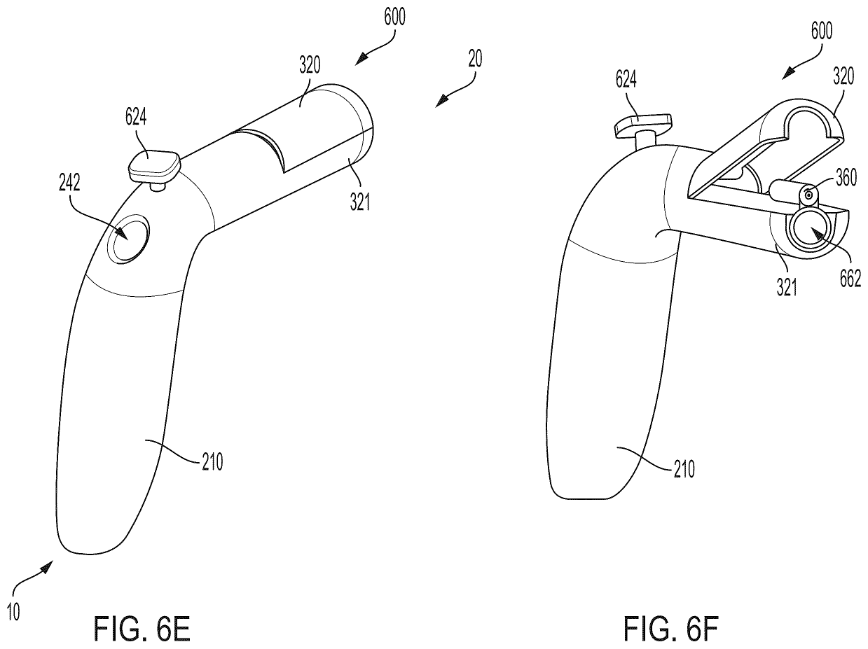

FIGS. 6A-F illustrate another embodiment of an access device 600 for use in a colposcopy or hysteroscopy procedure. The access device 600 has a proximal end 10 and a distal end 20.

As shown in FIGS. 6A-B, the first elongated member 202 of the proximal end 10 forms a handle 210 having a first elongated member central axis 11. The second elongated member 204 has a rotatable member 320. The rotatable member 320 is positioned distal to the handle 210. The rotatable member 320 can have a flat or substantially flat distal end 628 as illustrated. A distal end diameter controller 624 is provided on the handle 210 that is operable to rotate the rotatable member 320 at a hinge 327 away from the non-rotatable member 321 with a second elongated member central axis 21. The distal end diameter controller 624 can be a push button or a rotational button as illustrated. A wedge shaped gap 626 with a wider upper portion 627 of the wedge shaped gap 626 along the top of the second elongated member 204 from a side view of the access device 600 and a smaller gap near the pivot point 392 of the second elongated member 204. The wedge shaped gap 626 can be provided between the rotatable member 320 and an upper housing surface 251 of a section of the second elongated member 204. FIGS. 6C-D illustrate a side view and a front view (from the distal perspective) of the access device 600 with the rotatable member 320 extended away from the second elongated member central axis 21. When the rotatable member 320 extends away from the central axis, the shape of the wedge shaped gap 626 changes so that the space forming the gap along the top portion of the second elongated member decreases as the rotatable member 320 rotates away from the second elongated member central axis 21. FIG. 6E is a proximal perspective view showing the proximal housing aperture 242. From the distal perspective view in FIG. 6F, and integrated camera 360 is visible with a channel 662, such as an interior channel, below the camera.

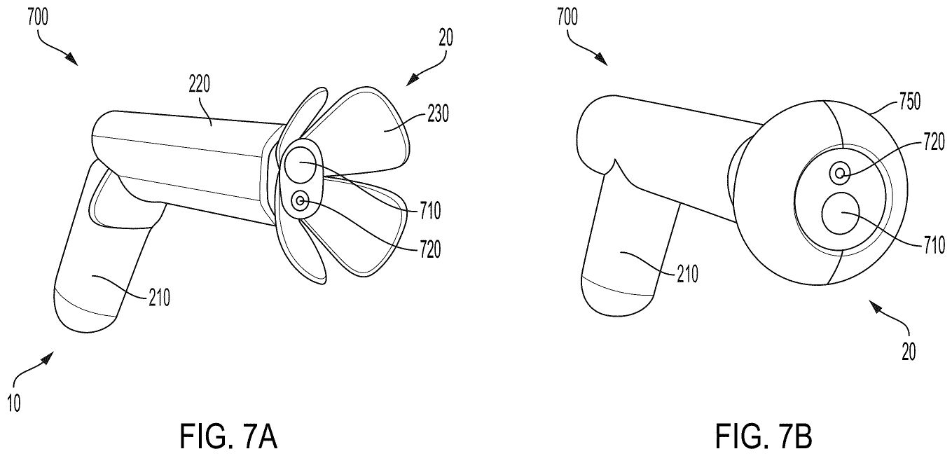

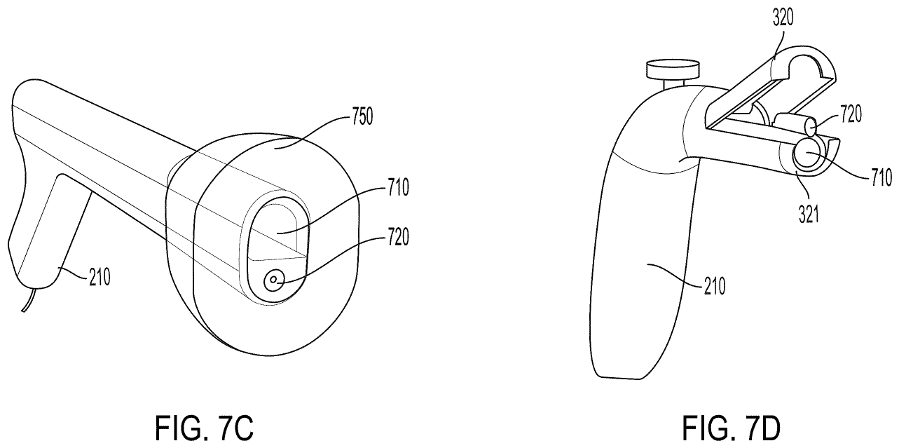

FIGS. 7A-D illustrate a variety of distal end configurations for the disclosed access devices described herein (e.g., access devices 200, 300, 400, 500, 600, 900, 1000) for use in a colposcopy or hysteroscopy procedure. From the distal end 20 one or more channels 710 can be present. An image capture device such as camera 720 can be incorporated into the access device or advanced through the device via a channel (e.g., as shown in FIG. 2N). As shown in FIG. 7A, two or more curved members 230 can be provided that are operable to extend away from a central axis during operation (e.g., when a retractable sheath is moved proximally). The two or more curved members (shown as four members) have non-parallel sides that curve toward a distal side. The curved members can have a petal-like shape and open in a flower-like arrangement. In another configuration, shown in FIGS. 7B and 7C, a distal inflatable member 750 can be provided that is operable as an expandable distal end. During insertion of the access device the distal inflatable member 750 is in an uninflated configuration, once inserted, the distal inflatable member 750 can be inflated to increase the distal diameter of the access device to, for example, expand the diameter of the vaginal opening to provide better visibility to the cervix. The inflatable member can be substantially round as shown in FIG. 7B, or oval as shown in FIG. 7C. FIG. 7D illustrates an access device with a rotatable member (similar to FIGS. 5-6), and an enclosed channel 710.

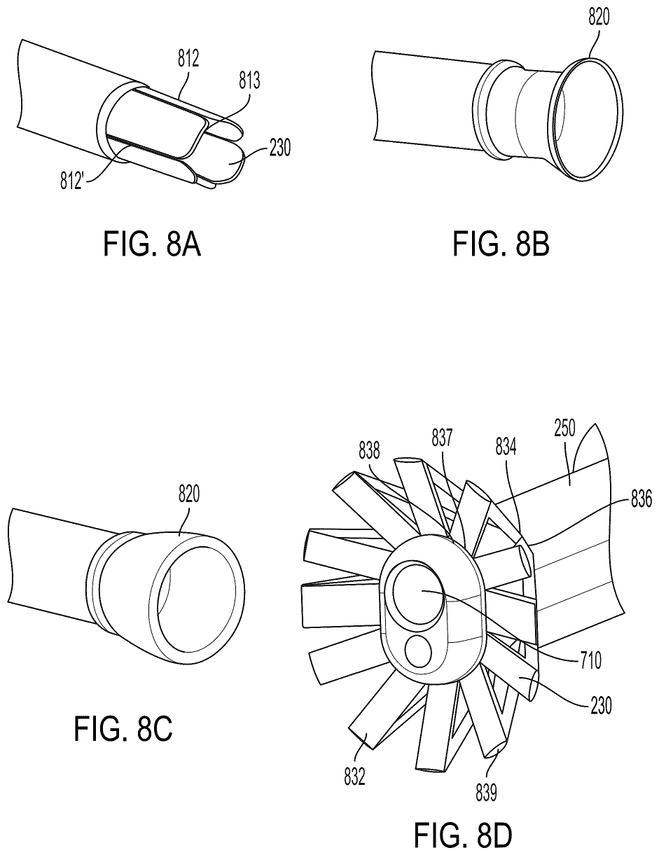

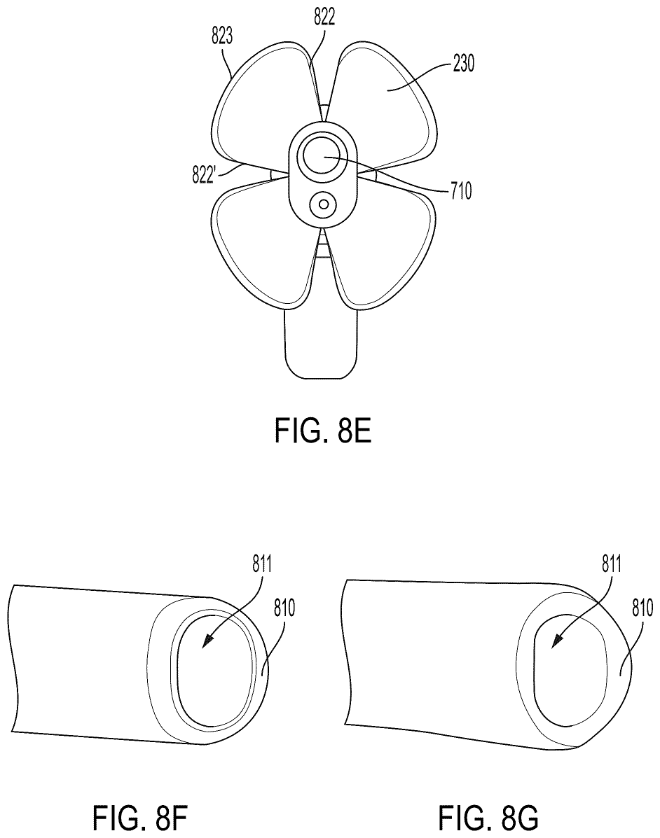

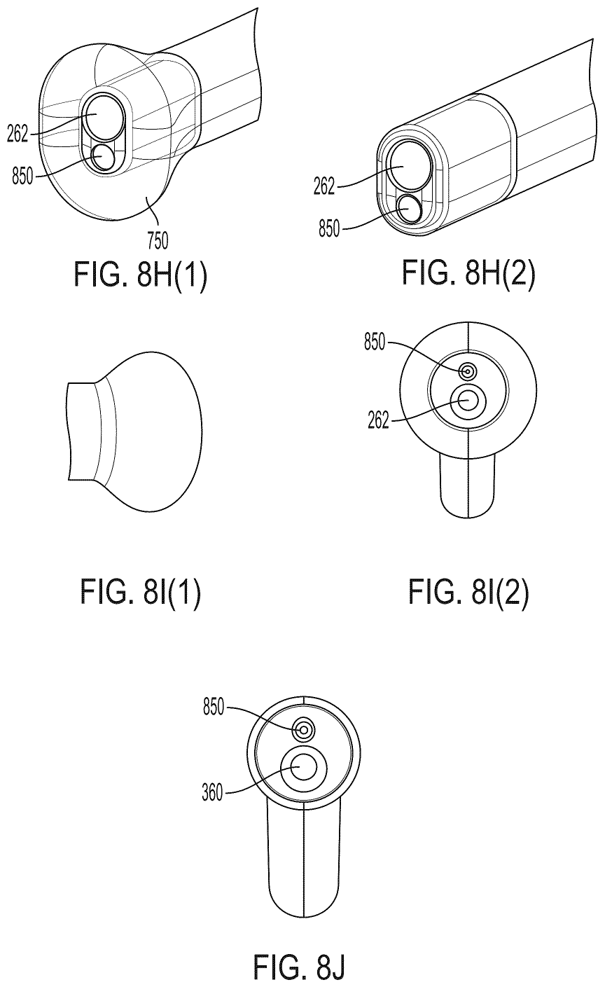

FIGS. 8A-G illustrate additional distal end configurations for the disclosed access devices for use in a colposcopy or hysteroscopy procedure. FIG. 8A illustrates two or more curved members 230 that are operable to extend away from a central axis during operation (e.g., when a retractable sheath is moved proximally) which can open in a flower-like arrangement. The curved members in this configuration have curved member parallel sides 812, 812′ perpendicular to a curved member distal edge 813. The curved member parallel sides 812, 812′ curve into the curved member distal edge 813, similar to three sides of a rounded rectangle. FIGS. 8B-C illustrate an additional configuration of an access device with a radially expanding distal end 820. The radially expanding distal end 820 can, for example, be configured similar to a drywall anchor so that the radially expanding distal end 820 increases in diameter when expanded after insertion of the access device. FIG. 8D illustrates an oval shaped distal end 830 with an access device channel and a built-in access device tool (e.g., camera or illumination device) with a plurality of rotatable members 832 operable to extend away from the central axis. The plurality of rotatable members 832 are curved rotatable members with parallel rotatable member sides perpendicular to a distal perpendicular member side. The curved members have a first curved member end 834 attached to an exterior of the housing 250 at a first location 836 and a second curved member end 837 attached to an exterior of the housing 250 at a second location 838 distal to the first location 836. When the curved rotatable members are expanded away from the second elongated member central axis 21, the first curved member end 834 extends away from the second curved member end 834′, and the curved member lengthens as a first half of the curved member pivots away from a second half of the curved member at a flexible joint 839. FIG. 8E illustrates four separate curved members 230 that are operable to extend away from a second elongated member central axis 21 during operation (e.g., when a retractable sheath is moved proximally). The curved members in this configuration have non-parallel sides 822, 822′ at an angle to the distal edge 823. FIGS. 8F-G illustrate a distal end of a sheath 810, such as a flexible sheath, for use with the disclosed access devices with an aperture 811. FIGS. 8H(1)-8H(2) illustrate a distal end of an access device as disclosed with a sheath 810 that is inflatable. FIGS. 8H(1)-8H(2) also show a working channel 262 positioned above a built in tool 850 (e.g., light or camera). As shown in FIGS. 8I(1)-8I(2) a working channel 262 can be positioned below a built in tool 850 (e.g., illumination device or camera). In yet another alternative, as shown in FIG. 8J, the camera 360 and light 860 can be fully built in or integrated into the access device.

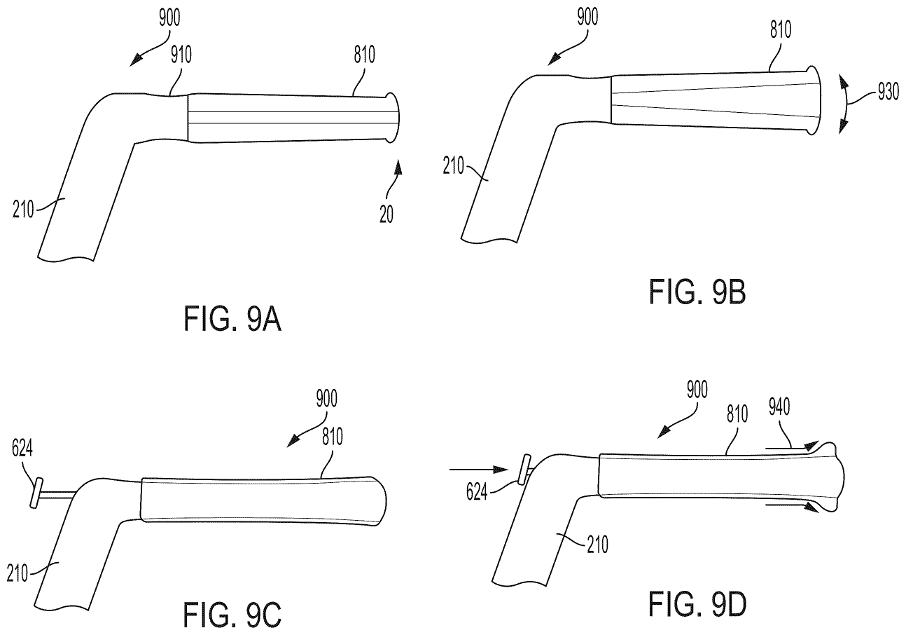

FIGS. 9A-D illustrate a sheath 810 that is inflatable positioned about the second elongated member of an access device 900 for use in a colposcopy or hysteroscopy procedure. FIG. 9A is a side view of an access device 900 with a slimmer section 910 (i.e., section with a smaller diameter than the diameter at the distal end 20) for the opening of the vagina. The sheath 810 is positioned distal to the slimmer section 910. As shown in FIG. 9B, in use the sheath 810 can be expanded 930 via an inner mechanism controllable by the distal end diameter controller. FIG. 9C illustrates a distal end diameter controller 624 (such as distal end diameter controller 624 from FIG. 6) which can be depressed (shown in FIG. 9D) towards the handle 210 to cause the sheath 810 to scrunch and increase in diameter 940 away from the central axis at the distal end.

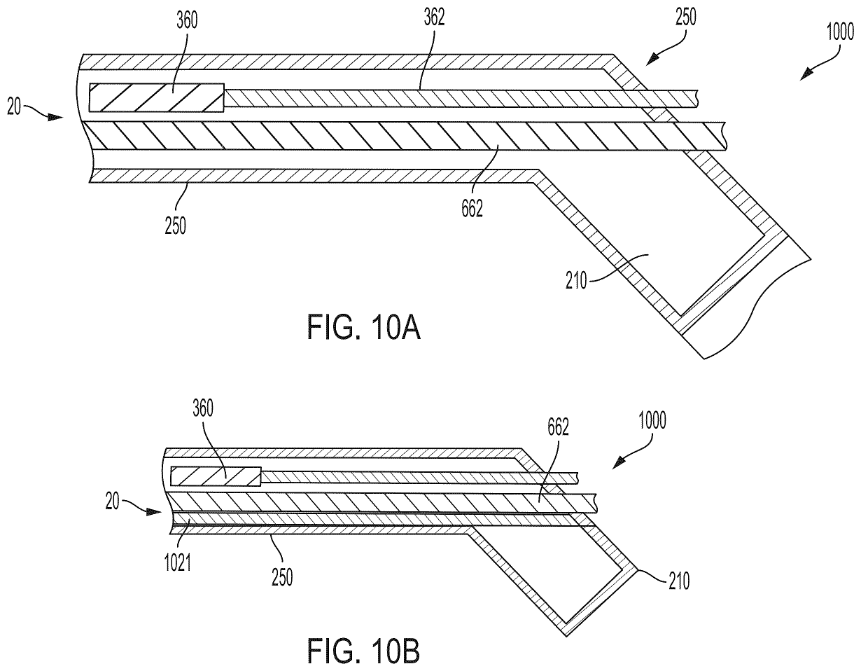

FIGS. 10A-B illustrate an exemplar cross-section for an access device 1000 for use in a colposcopy or hysteroscopy procedure. The cross-section shows a camera 360 connected to a cable or semi-rigid cable 362 positioned within the housing 250 of the access device 1000. A working channel 662 is shown positioned below the camera 360. The working channel can be defined by the housing (e.g. an interior cavity) or a separate structure positioned within the housing. In the configuration shown in FIG. 10B the camera 360 is shown above the working channel 662, and a second tool 1021 is shown in the housing 250 below the working channel 662. Other positions for the camera and working channel (e.g., side by side) can be used without departing from the scope of the disclosure.

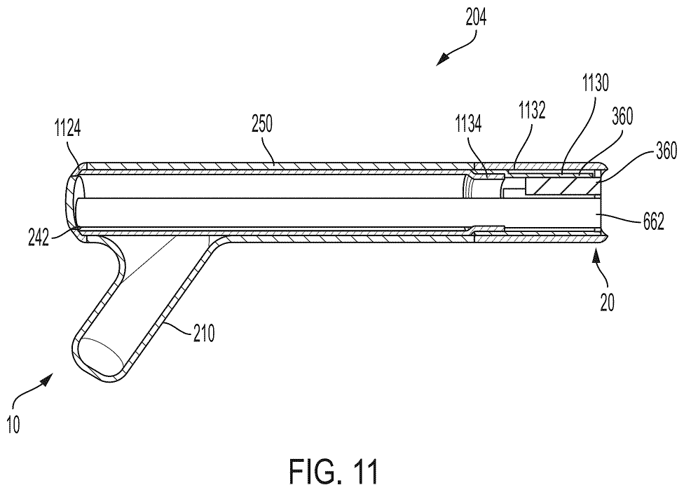

FIG. 11 illustrates another exemplar cross-section for an access device (e.g., access device 200, 300, 400, 500, 600, 900, 1000 disclosed herein) for use in a colposcopy or hysteroscopy procedure. The access device has a working channel 662 within the housing with an entrance at a proximal end of the second elongated member and an exit at a distal end of the second elongated member. A built-in camera 360 is shown positioned within the housing above the working channel 662. However, as will be appreciated by those skilled in the art, a built-in camera can be located below or beside the working channel without departing from the scope of the disclosure. Components of the housing comprising the second elongated member 204 can include a dial 1124, and a dial cap attached to the working channel at a proximal end 10, a collar 1130 with expandable springs at a distal end. The collar 1130 can have fixed end such that it is attached to the channel. A portion of the housing can be in threaded engagement with the collar. An elastomer 1132 can be provided that provides a smooth, uninterrupted exterior surface free of gaps or pinch points. Threads 1134 enable the tip feature to expand as shown in, for example, FIGS. 8D and 8H.

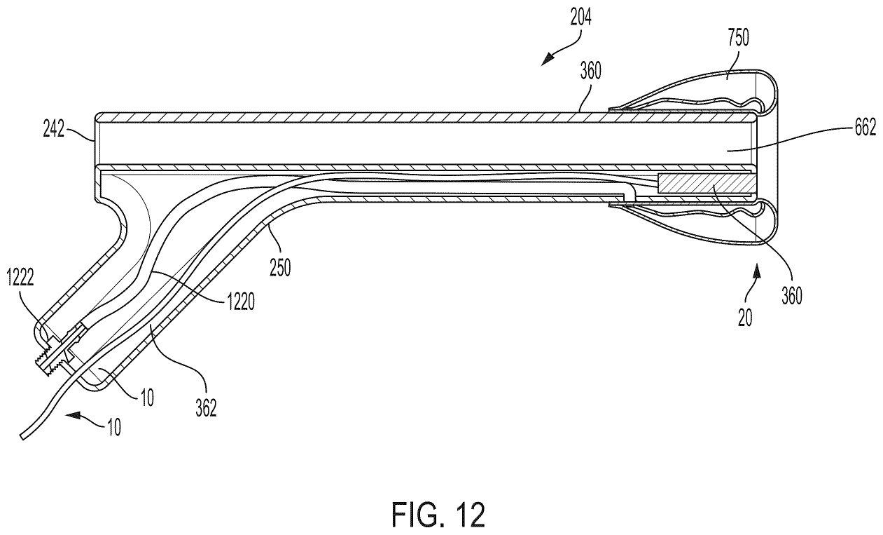

FIG. 12 illustrates another exemplar cross-section of an access device for use in a colposcopy or hysteroscopy procedure. In this configuration, the camera 360 is built in and positioned below the working channel 662. A distal inflatable member 750 is provided that is controllable by the user by use of an air hose 1220 positioned within the housing 1010. The air hose 1220 engages a syringe fitting 1222 that allows air or gas to pass through the hose to inflate the distal inflatable member 750. The build in camera 360 has wires or a semi-rigid cable 362 positioned within the housing.

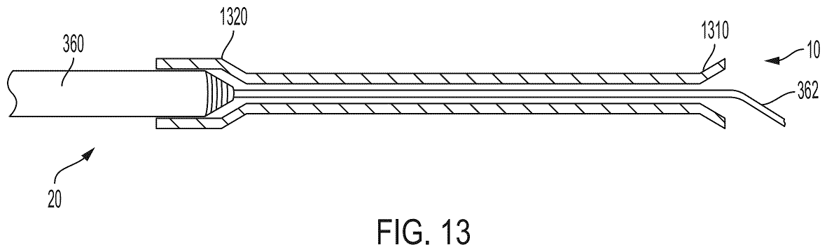

FIG. 13 illustrates another exemplar cross-section for an access device for use in a colposcopy or hysteroscopy procedure. In the configuration shown in FIG. 13, a push-pull point 1310 for advancing the camera 360 past the distal end 20 is shown. The camera 360 can be positioned within a hollow tube or channel with a larger diameter distal end operating as a camera mount 1320.

Turning now to FIGS. 14A-K, another access device 1400 is illustrated. The access device 1400 is configurable to operate as a speculum to provide better visualization and access inside the vagina. FIG. 14A is an isometric view of the access device 1400 with a housing 250 when a rotatable member 1420 and a non-rotatable member 1421 of a jaw 1401 at the distal end 20 are in a closed position. From the upper isometric view, the curved distal end of the housing 250 is visible. The rotatable member 1420 also has an opening 1422 along the top surface of the rotatable member 1420. Visible through the opening 1422 in the top surface are a right and left side of a tension wire 1482 positioned within an interior cavity of the housing. A release lever 1476 can be provided on the front surface 1410 of the handle. The release lever 1476 can be at least partially positioned within a protrusion 1477 that extends from the front surface 1410 of the handle 210. The protrusion 1477 can have a channel 1479 therein that allows the release lever 1476 surface to be continuous with the surface of the protrusion 1477 along at least a portion of the length of the release lever 1476. A protrusion recess 1481 that is sized and configured to allow a user to place a finger under the release lever 1476 and pull the release lever 1476 away from the housing 250.

FIG. 14B is an isometric view of the access device 1400 when the rotatable member 1420 and the non-rotatable member 1421 at the distal end 20 are in an open position. After insertion, the access device 1400 is activated so that the opening at the distal end of the access device increases in diameter. Increase in diameter can be achieved by, for example translating a rotatable member 1420 away from a non-rotatable member 1421 so that a proximal rotatable member surface 1420′ abutting a distal handle surface 1413 moves away from the distal handle surface 1413. As shown in FIG. 14A the proximal rotatable member surface 1420′ is parallel to the distal handle surface 1413. Once activated, as shown in FIGS. 14B-14C, the proximal rotatable member surface 1420′ is no longer parallel to the distal handle exterior surface 1411. Additionally, the tension wire 1482 that had been positioned within the interior cavity of the housing, is exterior to the upper exterior surface of the housing.

FIG. 14C is an isometric view from the proximal end 10 of the access device 1400 showing the rotatable member 1420 and the non-rotatable member 1421 at the distal end 20 in an open position. Also shown is a housing aperture 1442. The housing aperture 1442 is elongated with a conical shape (e.g., forming a conical elongated housing aperture) wherein a first diameter (positioned nearest the handle) has a larger diameter than a second diameter (positioned closest the distal end). The conical shape of the housing aperture 1442 facilitates an easy pass-through of standard procedural instrumentation during use and a sweep of a larger anatomical area by, for example, a tissue collection device. Also visible in FIG. 14C is the distal end diameter controller 1425 or button. The distal end diameter controller 1425 has a diameter controller upper surface 1426 that is angled to prevent the distal end diameter controller 1425 from impeding rotation of standard procedural instrumentation during use via the housing aperture 1442 during use.

FIG. 14D is an exploded view of the access device 1400. The access device 1400 can be provided with video visualization as illustrated in FIG. 14D. In this view, a general assembly of the access device 1400 is illustrated including the camera 1460. The camera 1460 is first attached to a camera mount 1462. This camera assembly is then attached to the underside of the tension wire 1482 and capped with a camera mount cap 1464 on the outer side of the tension wire 1482. The camera mount 1462 has an angular geometry operable to direct the camera 1460 towards a target surface for optimal visualization by the user. The camera 1460 can also have a camera cable 1466 that connects the camera 1460 to electronics in the access device 1400 (e.g., in the handle) or to a remote device (e.g., tablet). Also shown in this view is a method to attach the tension wire 1482 to the jaw 1401. The tension wire 1482 slides into a groove 1483 of the rotatable member 1420 of the jaw 1401 then a jaw wire cap 1468 attaches to the jaw 1401 securing the tension wire 1482 but still allowing pivoting of the rotatable member 1420 relative to the non-rotatable member 1421. The distal end diameter controller 1424 can have a series of notches or teeth (now shown) formed in the lower surface 1426 of the distal end diameter controller 1425. As described above, the upper surface can have an angled surface 1427 which allows the access device 1400 to accommodate the rotation of a standard procedural instrument during use. The handle front 1410 has a release lever 1476 secured to a release lever spring 1474.

FIG. 14E is a cross-section view of the access device 1400 in the closed position with the gap between rotatable member 1420 and the non-rotatable member 1421 at the distal end 20 at the smallest distance. The distal end diameter controller 1424, such as a control button, is in a fully extended position, i.e. having an interface surface positioned away from back exterior surface 1411 of the handle 210 allowing, the rotatable member 1420 to be in the closed position. The closed position is the configuration of the access device 1400 used during insertion.

FIG. 14F is a cross-section view of the access device 1400 in the open position. The distal end diameter controller 1424 is in the fully depressed position which pushes a control wire 1472 distally activating the jaw 1401 so that the rotatable member 1420 translates away from the non-rotatable member 1421, thereby opening or widening the distal end of the access device 1400. In the open position, the camera 1460 now has visualization of the target anatomy. Additionally, this configuration allows a user to push (e.g., provide distal pressure) on the distal end diameter controller 1424 to open the jaw without requiring any other unlocking maneuver. A release lever spring 1474 engages a release lever 1476 provided on an exterior surface (handle front surface 1410) of the handle 210. The release lever 1476 allows the distal end diameter controller 1424 to travel distally during actuation by the user, opening the jaw 1401 but provides enough resistance against the jaw 1401 closing to maintain the jaw 1401 in an open configuration. To close the jaw 1401, the release lever 1476 is depressed (e.g., force applied to move the release level towards the exterior surface of the handle), allowing the distal end diameter controller 1424 to be retracted. Retracting the distal end diameter controller 1424 also retracts the control wire 1478 allowing the jaw to return to the closed position by moving the rotatable member 1420 towards the non-rotatable member 1421. When the release lever 1476 is actuated, the jaw 1401 can slowly close by application of pressure onto the exterior of the housing during the removal process.

FIG. 14G is a cross-sectional view of the access device 1400 showing the release lever 1476 depressed (i.e., positioned towards the exterior surface of the handle) allowing jaw closure with distal end diameter controller 1424 retraction. As illustrated, the control wire 1478 is positioned adjacent an opposing interior surface of the housing 250 as the tension wire 1482.

FIG. 14H is a cross-section view of the access device 1400 in the open position with the rotatable member 1420 of the jaw translated away from the non-rotatable member 1421. The jaw opens when the distal end diameter controller 1424 is depressed advancing the control wire 1478 which, in turn, advances the jaw into the open configuration. The jaw-control wire pivot joint 1480 allows the jaw to advance linearly toward the distal direction and also pivot. A tension wire 1482 is provided that is operable to maintain a distance from the jaw-tension wire joint 1484 and the handle-tension wire joint 1486. Maintaining the distance between the jaw-tension wire joint 1484 and the handle-tension wire joint 1486 and articulating the jaw as illustrated keeps the distal tip of the jaw in a distal location which is advantageous for cervical examination.

FIGS. 14I-14J illustrate an articulating jaw using one of two methods. FIG. 14I illustrates a standard way of pivoting the rotatable member 1420 from the bottom creating a distance d1 between the distal end of the rotatable member 1420 and the distal end of the non-rotatable member 1421. FIG. 14J illustrates a linear translation and pivoting of the rotatable member 1420 on the bottom with the tension wire 1482 substantially maintaining the distance d2 between the distal end of the rotatable member 1420 and the distal end of the non-rotatable member 1421. The result of the method on the right is that the jaw tip travels much less in the proximal direction maintaining its effectiveness at the distal end of the access device 1400. Additionally, the barrel 1490 of the access device 1400, i.e., the portion of the access device housing that includes the rotatable member 1420 and the non-rotatable member 1421 has three separate zones with different diameters. The distal most portion of the barrel 1491 has a first diameter in an undeployed configuration with a sloping surface towards the distal tip 1494. The distal most portion first diameter is variable and increases as the access device is deployed. Proximal to the distal most portion of the barrel, the intermediate portion 1492, has a second diameter that is less than the largest diameter of the distal most portion of the barrel. The second diameter is a fixed diameter. Proximal to the intermediate portion 1492, is the proximal portion of the barrel 1493 which has a third diameter greater than the second diameter of the intermediate portion. The cross-section of the barrel is circular. During use, the smaller diameter of the intermediate portion 1492 allows the user to insert the access device 1400 and then rotate the device 45 to 900 with reduced discomfort for the patient.

The distal housing portion 1491, e.g. distal portion of the barrel of the access device, has a closed configuration width of 25 mm to 35 mm, more preferably 31 mm, and a height of 20 mm to 30 mm, more preferably 24 mm, and an open figuration diameter of from 40 mm to 60 mm, more preferably 48 mm. The length of the distal portions is from 50 mm to 70 mm, more preferably 57 mm. The intermediate housing portion 1492 can have the same length and width as the proximal section 1491 or could have a diameter of from 20 mm to 30 mm, more preferably 24 mm and a length of from 25 mm to 35 mm, more preferably 30 mm. The proximal housing portion 1493 has a cross-sectional diameter of from 30 mm to 40 mm, more preferably 36 mm and a length of from 20 mm to 35 mm, more preferably 27 mm.

FIG. 14K illustrates the access device 1400 with a sample collection instrument 1408.

### II. Methods

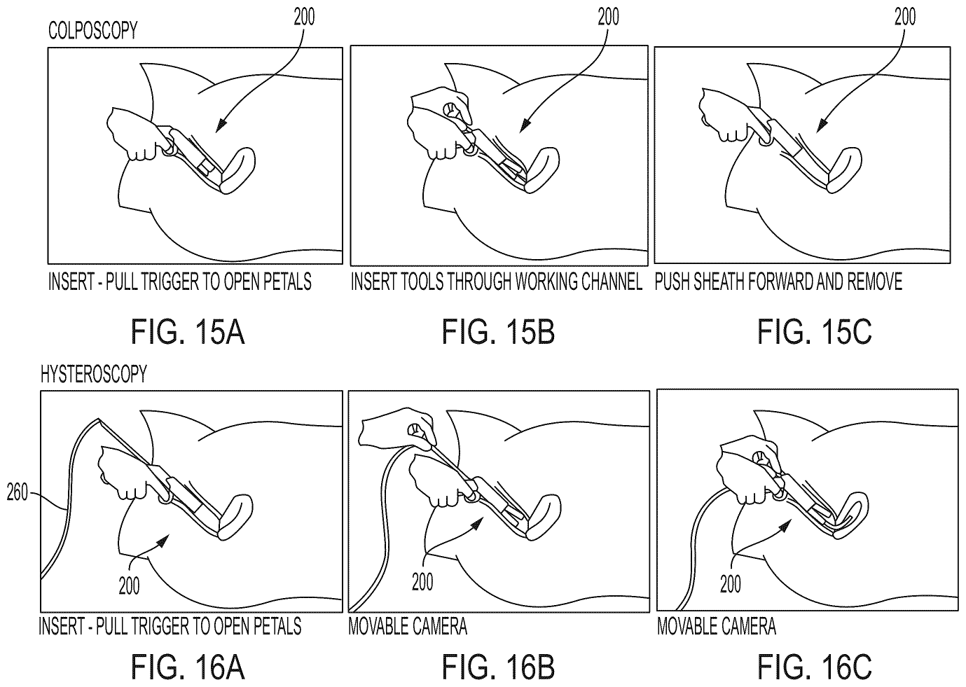

FIGS. 15-C illustrates steps for performing a colposcopy using an access device 200, 300, 400, 500, 600, 900, 1000,1400 as disclosed herein. The access device and methods can be used in a remote or mobile location, including at home. Additionally, the access device and methods can be used by the person directly or with the assistance of a trained or untrained person.

In FIG. 15A, an access device 200, 300, 400, 500, 600, 900, 1000, 1400 as disclosed herein is inserted into the vagina. Once inserted the access device may be rotated. The access device is then activated using the distal end diameter controller (e.g., pulling trigger, pressing, rotating or sliding the button). Once the access device is activated, if the tools are not built in, one or more tools can be inserted through one or more working channels as shown in FIG. 15B. The healthcare practitioner then views the cervix and surrounding tissues. A pap smear can be performed to collect tissue samples. Once the procedure is completed the access device can be deactivated and withdrawn (FIG. 15C). If the access device includes an expandable sheath, the controller is released to allow the sheath to encircle the distal end prior to withdrawal.

FIGS. 16A-C illustrates steps for performing a hysteroscopy using an access device 200, 300, 400, 500, 600, 900, 1000, 1400 as disclosed herein. In FIG. 16A, an access device as disclosed herein is inserted into the vagina. The access device is activated using the controller (e.g., pulling trigger). Once the access device is activated, a moveable camera can be inserted through a working channel as shown in FIG. 16B. Once the procedure is completed the access device can be deactivated and withdrawn (FIG. 16C).

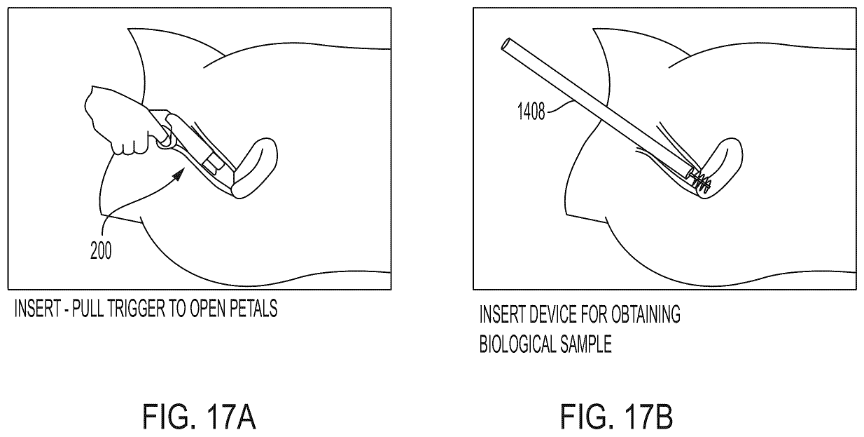

FIGS. 17A-B illustrate steps for obtaining a biological sample using an access device 200, 300, 400, 500, 600, 900, 1000, 1400 as disclosed herein. In FIG. 17A, an access device as disclosed herein is inserted into the vagina. The access device is activated using the controller (e.g., pulling trigger). Once the access device is activated, sample collection device 1408 (e.g., surgical tools for performing biopsies) can be inserted to obtain a biological sample as shown in FIG. 17B. Once the procedure is completed the access device can be deactivated and withdrawn.

For any of the methods, a photo or image can be taken (e.g., of the cervix, vagina or biological sample) by the camera associated with the access device or by an electronic device having an app downloadable thereon (e.g., mobile phone or tablet). The camera can provide a zoom function that allows one or more close-up images of a target anatomy or tissue. More than one image can be captured at different times or occasions or of the same scene (anatomy) or different scenes. The photo or image can be processed by the app or transmitted to a remote location for further evaluation. Comparison of images to identify changes between images can be performed.

### III. Kits

Kits can include an access device 200, 300, 400, 500, 600, 900, 1000, 1400 for performing a colposcopy and/or hysteroscopy procedure as disclosed herein. In addition to the access device for performing a colposcopy and/or hysteroscopy device, one or more tools can be provided including, but not limited to a camera, an endocervical curette, a cervical biopsy pump, cervical swabs, a sample collection device, and vials for storing collected materials.

Additionally, kits can include one or more items for at home or telehealth usage. For example, one or more swabs operable to detect one or more target virus or bacteria. The swabs can have an elongated member suitable to be held by a user at a first end and a second end operable to obtain a sample, e.g. with the use of bristles. Target virus or bacterial to be detected includes, for example, aerobic vaginitis (AV), e.g. E. coli, group B streptococci, bacterial vaginosis (BV), e.g., Gardnerella vaginalis, Atopobium vaginae, Prevotella bivia, cytolytic vaginosis (CV), e.g., Lactobacillus crispatus, STIs, e.g., chlamydia, gonorrhea, trich, Mycoplasma genitalium, Lactobacillus crispatus, Lactobacillus gasseri, staphylococcus, aerococcus, Candida albicans, Candida glabrata, Mycoplasma hominis, Ureaplasma parvum, and Ureaplasma urealyticum.

### IV. Software, Computing and Network Environments

As part of the system for using a disclosed access device 200, 300, 400, 500, 600, 900, 1000, 1400 a software application (“app”) can be provided and downloaded to a memory on an electronic device or stored on a memory associated with the IUD removal device. The software can be operable to provide audible instructions and/or visual instructions (static (e.g. pictures), non-static (e.g. video), or a combination thereof). Additionally, the software can include artificial intelligence (AI) capabilities including, for example enabling the computing device to perform a variety of advanced functions, including the ability to see, understand and translate spoken and written language, analyze data, make recommendations, and more. The software can also be operable to detect abnormalities in an image acquired by the image capture device. Additionally, the AI capability can be used for image analysis of an image taken by a user when using the disclosed devices, generating predictions, and generating an AI-assisted diagnosis and treatment recommendation for a patient.

The memory, which may be internal memory or external memory to a microcontroller, may be implemented in firmware and/or software implementation. The firmware and/or software implementation methodologies may be implemented with modules (e.g., procedures, functions, and so on) that perform the functions described herein. A machine-readable medium tangibly embodying instructions may be used in implementing the methodologies described herein. For example, software codes may be stored in a memory and executed by a processor unit (e.g., microcontroller). Memory may be implemented within the processor unit or external to the processor unit. As used herein, the term “memory” refers to types of long term, short term, volatile, nonvolatile, and/or other non-transitory memory and is not to be limited to a particular type of memory or number of memories, or type of media upon which memory is stored.

If implemented in firmware and/or software, and/or as part of microcontroller and/or memory, the functions described herein may be stored as one or more instructions or code on a computer-readable medium. Examples include computer-readable media encoded with a data structure and computer-readable media encoded with a computer program. Computer-readable media includes physical computer storage media. A storage medium may be an available medium that can be accessed by a computer. By way of example, and not limitation, such computer-readable media can include RAM, ROM, EEPROM, CD-ROM or other optical disk storage, magnetic disk storage or other magnetic storage devices, or other medium that can be used to store desired program code in the form of instructions or data structures and that can be accessed by a computer (e.g., microcontroller); disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors (e.g., microcontroller) to implement the functions outlined. The communication components enable wired or wireless transmission of control commands from the control switches on the device in order to control one or more of operation software, and/or image processing software, transmission of control commands from the image processing software, and transmission of data.

Memories are configurable to store data within computing devices. In one implementation, memory is a volatile memory unit or units. In another implementation, memory is a non-volatile memory unit or units. Memory can also be another form of computer-readable medium (e.g., a magnetic disk, optical disk or solid state disk). Memory can also be non-transitory.

Storage devices are capable of providing mass storage for computing device. In one implementation, storage device can be or contain a computer-readable medium (e.g., a floppy disk device, a hard disk device, an optical disk device, or a tape device, a flash memory or other similar solid state memory device, or an array of devices, such as devices in a storage area network or other configurations). A computer program product can be tangibly embodied in a data carrier. The computer program product also can contain instructions that, when executed, perform one or more methods (e.g., those described above.) The data carrier is a computer- or machine-readable medium, (e.g., memory, storage device, memory on processor, and the like).

High-speed controllers manage bandwidth-intensive operations for computing device, while low speed controllers manage lower bandwidth-intensive operations. Such allocation of functions is an example only. In one implementation, high-speed controller is coupled to memory, display (e.g., through a graphics processor or accelerator), and to high-speed expansion ports, which can accept various expansion cards. In the implementation, low-speed controllers are coupled to storage devices and low-speed expansion port. The low-speed expansion port, which can include various communication ports (e.g., USB, Bluetooth®, Ethernet, wireless Ethernet), can be coupled to one or more input/output devices (e.g., a keyboard, a pointing device, a scanner, or a networking device including a switch or router, e.g., through a network adapter). Computing devices can be implemented in a number of different forms, as shown in the figure. For example, computing devices can be implemented as standard server, or multiple times in a group of such servers. Computing devices can be implemented as part of rack server system. In addition, or as an alternative, it can be implemented in a personal computer (e.g., laptop computer). In some examples, components from computing devices can be combined with other components in a mobile device (not shown), e.g., device. Each of such devices can contain one or more of computing devices and an entire system can be made up of multiple computing devices communicating with each other.

Computing device includes processor, memory, an input/output device (e.g., display, communication interface, and transceiver) among other components. Device also can be provided with a storage device, (e.g., a micro drive or other device) to provide additional storage. Each of the devices, processor, display, memory, communication interfaces, and transceiver, are interconnected using various buses, and several of the components can be mounted on a common motherboard or in other manners as appropriate. The software can be in communication with one or more of a database or data bank of images.

As will be appreciated by those skilled in the art, the present teachings may also extend to one or more of the following numbered clauses:

1. A device comprising: a housing having a proximal housing end and a distal housing end, wherein a first portion of the housing extending from the proximal housing end defines a first elongated member and a second portion of the housing extending from the first elongated member to the distal housing end fines a second elongated member; one or more channels defined within an interior of the housing; an expandable distal housing end; a distal end diameter controller wherein the distal end diameter controller is operable to control a diameter of a distal end of the housing; and a conical elongated housing aperture with a first conical elongated housing aperture diameter and a second conical elongated housing aperture diameter, larger than the first conical elongated housing aperture diameter.

2. A device comprising: a housing having a proximal housing end and a distal housing end, wherein a first portion of the housing extending from the proximal housing end defines a first elongated member and a second portion of the housing extending from the first elongated member to the distal housing end fines a second elongated member wherein the first elongated member has an expandable distal housing portion having a first diameter in a closed configuration, a proximal housing portion having a second diameter and an intermediate housing portion positioned between the distal housing portion and the proximal housing portion having a third diameter less than the first diameter; one or more channels defined within an interior of the housing; a distal end diameter controller wherein the distal end diameter controller is operable to control a diameter of a distal end of the housing; and a housing aperture positioned operable to provide access to an interior of the housing.

3. A device comprising: a housing having a proximal housing end and a distal housing end, wherein a first portion of the housing extending from the proximal housing end defines a first elongated member and a second portion of the housing extending from the first elongated member to the distal housing end fines a second elongated member; one or more channels defined within an interior of the housing; a distal end diameter controller wherein the distal end diameter controller is operable to control a diameter of a distal end of the housing; an expandable distal housing end; and one or more electronic components operable to transmit information captured by the device to a secondary electronic device.

4. A device comprising: a housing having a proximal housing end and a distal housing end, wherein a first portion of the housing extending from the proximal housing end defines a first elongated member and a second portion of the housing extending from the first elongated member to the distal housing end fines a second elongated member; one or more channels defined within an interior of the housing; a distal end diameter controller wherein the distal end diameter controller is operable to control a diameter of a distal end of the housing and further wherein the distal end diameter control is selected from a rotatable member, a slidable button, a pressable button, a retractable sheath trigger and a retractable s heath lever; and an expandable distal housing end.

5. The device of numbered clause 1 or 2 further comprising a tension wire positioned within the housing.

6. The device of numbered clause 1 or 2 further comprising a jaw control wire.

7. The device of numbered clause 1 further comprising a release lever.

8. The device of numbered clause 7 further comprising a release lever spring in communication with the release lever.

9. The device of numbered clause 7 further wherein the release lever spring is positioned within a channel formed in a housing protrusion.

10. The device of numbered clause 7 further comprising a housing recess under the release lever.

11. The device of numbered clause 1 or 2 wherein the distal end diameter controller has an exterior controller surface with a first controller surface angularly positioned to a second controller surface.

12. The device of any of numbered clauses 1-4 further comprising one or more image capture devices with an associated optical lens or lens system, wherein the one or more image capture devices are positionable within the housing and operable to collect one or more images of a visual field located outside the distal housing end.

13. The device of numbered clause 12, wherein the associated optical lens or lens system has a lens chosen from: a narrow field of view lens; a fish-eye lens; an omni-directional lens; and a lens having zoom-in and zoom-out functions.

14. The device of numbered clause 12, wherein the one or more image capture devices is operable to acquire images selected from: still images and video images.

15. The device of any of numbered clauses 1-4 further comprising one or more image capture devices with an associated optical lens or lens system, wherein the one or more image capture devices are removably positionable within the housing via a proximal housing aperture and operable to collect one or more images of a visual field located outside the distal housing end.

16. The device of any of numbered clauses 1-4 further comprising an illumination assembly operable to illuminate a visual field located outside the distal housing end.

17. The device of numbered clause 16 wherein the illumination assembly is removably positioned within the housing at a distal housing end via a proximal housing aperture.

18. The device of numbered clause 16, wherein the illumination assembly is operable to deliver illumination in at least one of visible, near infrared, infrared, or ultraviolet spectral regions.

19. The device of numbered clause 16, wherein the illumination assembly includes a light source chosen from: Light Emitting Diodes, and an external illumination source insertable into a channel and operable to generate light.

20. The device of numbered clause 16, wherein the illumination assembly provides illumination in an illumination spectral range substantially the same as an image capture sensitivity spectral range.

21. The device of any of numbered clauses 1-4, wherein the one or more channels are operable to allow passage of one or more of: medicinal fluids; fluids for cleaning the interior of a vaginal canal; fluids for cleaning an exterior of a cover; gas for inflating inner spaces of a body; surgical tools for collecting biological samples; cameras; lights; and sample collection devices.

22. A system comprising software operable to: communicate with a device of any of numbered clauses 1-21; control an operation of the device; capture one or more images of interest via an image capture device and store the one or more images of interest in a database; and control a zoom function of an optical lens.

23. A system according to numbered clause 22, comprising image processing software operable to: process the images acquired by an image capture device; detect abnormalities in an image acquired by the image capture device; and perform image comparison to detect changes between images taken at different locations, between images of a same scene captured at different occasions, or between an image and images in a data bank.

24. A system according to numbered clause 23, wherein a communication component provides communications between the device and a computer and/or display, further wherein the communication components enable transmission of control commands from the control switches on the device in order to control one or more of operation software, and/or image processing software, and transmission of control commands from the image processing software.

25. A system according to numbered clause 22, wherein one or more control commands are transmitted by one or more communication components using one or more of: wireless communication, transmission of optical signals through fiber optic cables, and transmission of electrical signals through wires or cables.

26. A method of using an access device comprising: providing the device of any of numbered clauses 1-21; inserting the device into a vagina; controlling an operation of the device via a distal end diameter controller; and activating a tool.

27. The method of numbered clause 26 further comprising: controlling the operation of the access device by at least one of pulling a trigger, pressing a button, rotating a button, or sliding a button.

28. The method of numbered clause 26 further comprising: advancing a tool through a channel in the access device.

29. The method of numbered clause 26 further comprising: capturing one or more images of interest via an image capture device; storing the one or more images of interest in a data base; and controlling a zoom function of an optical lens.

30. The method of numbered clause 26 further comprising: obtaining a biological sample.

31. The method of numbered clause 26 further comprising: applying an illumination to a target surface exterior to the distal housing end.

32. A kit comprising: a device of any of numbered clauses 1-21; and one or more of a camera, an endocervical curette, a cervical biopsy pump, cervical swab, sample collection device, and vial.

While preferred embodiments of the present invention have been shown and described herein, it will be obvious to those skilled in the art that such embodiments are provided by way of example only. Numerous variations, changes, and substitutions will now occur to those skilled in the art without departing from the invention. For example, the use of comprise, or variants such as comprises or comprising, includes a stated integer or group of integers but not the exclusion of any other integer or group of integers. It should be understood that various alternatives to the embodiments of the invention described herein may be employed in practicing the invention. It is intended that any claims presented define the scope of the invention and that methods and structures within the scope of these claims and their equivalents be covered thereby.

What is claimed:

1. A device comprising:
a housing having a proximal housing end and a distal housing end, wherein a first portion of the housing extending from the proximal housing end defines a first elongated member and a second portion of the housing extending from the first elongated member to the distal housing end fines a second elongated member;
one or more channels defined within an interior of the housing;
an expandable distal housing end;
a distal end diameter controller wherein the distal end diameter controller is operable to control a diameter of a distal end of the housing; and
a conical elongated housing aperture with a first conical elongated housing aperture diameter and a second conical elongated housing aperture diameter, larger than the first conical elongated housing aperture diameter.

2. The device of claim 1 further comprising a tension wire positioned within the housing.

3. The device of claim 1 further comprising a jaw control wire.

4. The device of claim 1 further comprising a release lever.

5. The device of claim 4 further comprising a release lever spring in communication with the release lever.

6. The device of claim 4 further wherein the release lever spring is positioned within a channel formed in a housing protrusion.

7. The device of claim 4 further comprising a housing recess under the release lever.

8. The device of claim 1 wherein the distal end diameter controller has an exterior controller surface with a first controller surface angularly positioned to a second controller surface.

9. The device of claim 1 further comprising one or more image capture devices with an associated optical lens or lens system, wherein the one or more image capture devices are positionable within the housing and operable to collect one or more images of a visual field located outside the distal housing end.

10. The device of claim 9, wherein the associated optical lens or lens system has a lens chosen from: a narrow field of view lens; a fish-eye lens; an omni-directional lens; and a lens having zoom-in and zoom-out functions.

11. The device of claim 9, wherein the one or more image capture devices is operable to acquire images selected from: still images and video images.

12. The device of claim 1 further comprising one or more image capture devices with an associated optical lens or lens system, wherein the one or more image capture devices are removably positionable within the housing via a proximal housing aperture and operable to collect one or more images of a visual field located outside the distal housing end.

13. The device of claim 1 further comprising an illumination assembly operable to illuminate a visual field located outside the distal housing end.

14. The device of claim 13 wherein the illumination assembly is removably positioned within the housing at a distal housing end via a proximal housing aperture.

15. The device of claim 13, wherein the illumination assembly is operable to deliver illumination in at least one of visible, near infrared, infrared, or ultraviolet spectral regions.

16. The device of claim 13, wherein the illumination assembly includes a light source chosen from: Light Emitting Diodes, and an external illumination source insertable into a channel and operable to generate light.

17. The device of claim 13, wherein the illumination assembly provides illumination in an illumination spectral range substantially the same as an image capture sensitivity spectral range.

18. The device of claim 1, wherein the one or more channels are operable to allow passage of one or more of: medicinal fluids; fluids for cleaning the interior of a vaginal canal; fluids for cleaning an exterior of a cover; gas for inflating inner spaces of a body; surgical tools for collecting biological samples; cameras; lights; and sample collection devices.

19. A device comprising:
a housing having a proximal housing end and a distal housing end, wherein a first portion of the housing extending from the proximal housing end defines a first elongated member and a second portion of the housing extending from the first elongated member to the distal housing end fines a second elongated member wherein the first elongated member has an expandable distal housing portion having a first diameter in a closed configuration, a proximal housing portion having a second diameter and an intermediate housing portion positioned between the distal housing portion and the proximal housing portion having a third diameter less than the first diameter;
one or more channels defined within an interior of the housing;
a distal end diameter controller wherein the distal end diameter controller is operable to control a diameter of a distal end of the housing; and
a housing aperture positioned operable to provide access to an interior of the housing.

20. A device comprising:
a housing having a proximal housing end and a distal housing end, wherein a first portion of the housing extending from the proximal housing end defines a first elongated member and a second portion of the housing extending from the first elongated member to the distal housing end fines a second elongated member;
one or more channels defined within an interior of the housing;
a distal end diameter controller wherein the distal end diameter controller is operable to control a diameter of a distal end of the housing;
an expandable distal housing end; and
one or more electronic components operable to transmit information captured by the device to a secondary electronic device.

21. A device comprising:
a housing having a proximal housing end and a distal housing end, wherein a first portion of the housing extending from the proximal housing end defines a first elongated member and a second portion of the housing extending from the first elongated member to the distal housing end fines a second elongated member;
one or more channels defined within an interior of the housing;
a distal end diameter controller wherein the distal end diameter controller is operable to control a diameter of a distal end of the housing and further wherein the distal end diameter control is selected from a rotatable member, a slidable button, a pressable button, a retractable sheath trigger and a retractable s heath lever; and
an expandable distal housing end.

22. A system comprising software operable to:
communicate with a device comprising
a housing having a proximal housing end and a distal housing end, wherein a first portion of the housing extending from the proximal housing end defines a first elongated member and a second portion of the housing extending from the first elongated member to the distal housing end fines a second elongated member,
one or more channels defined within an interior of the housing,
a distal end diameter controller wherein the distal end diameter controller is operable to control a diameter of a distal end of the housing and further wherein the distal end diameter control is selected from a rotatable member, a slidable button, a pressable button, a retractable sheath trigger and a retractable s heath lever, and
an expandable distal housing end;
control an operation of the device;
capture one or more images of interest via an image capture device and store the one or more images of interest in a database; and
control a zoom function of an optical lens.

23. A method of using an access device comprising:
providing the access device having
a housing having a proximal housing end and a distal housing end, wherein a first portion of the housing extending from the proximal housing end defines a first elongated member and a second portion of the housing extending from the first elongated member to the distal housing end fines a second elongated member,
one or more channels defined within an interior of the housing,
a distal end diameter controller wherein the distal end diameter controller is operable to control a diameter of a distal end of the housing and further wherein the distal end diameter control is selected from a rotatable member, a slidable button, a pressable button, a retractable sheath trigger and a retractable s heath lever, and
an expandable distal housing end;
inserting the access device into a vagina;
controlling an operation of the access device via a distal end diameter controller; and
activating a tool.
